SEQUENCE LISTING (53 sequences)

SEQ ID NO: 1 - protein, 37 aa, Homo sapiens
LLGDFFRKSKEKIGKEFKRIVQRIKDFLRNLVPRTES

SEQ ID NO: 2 - protein, 34 aa, Mus musculus
GLLRKGGEKIGEKLKKIGQKIKNFFQKLVPQPEQ

SEQ ID NO: 3 - protein, 30 aa, Homo sapiens
ACYCRIPACIAGERRYGTCIYQGRLWAFCC

SEQ ID NO: 4 - protein, 34 aa, Homo sapiens
VCSCRLVFCRRTELRVGNCLIGGVSFTYCCTRVD

SEQ ID NO: 5 - protein, 34 aa, Homo sapiens
ARATCYCRTGRCATRESLSGVCEISGRLYRLCCR

SEQ ID NO: 6 - protein, 33 aa, Homo sapiens
RAFTCHCRRSCYSTEYSYGTCTVMGINHRFCCL

SEQ ID NO: 7 - protein, 36 aa, Homo sapiens
DHYNCVSSGGQCLYSACPIFTKIQGTCYRGKAKCCK

SEQ ID NO: 8 - protein, 40 aa, Homo sapiens
IGDPVTCLKSGAICHPVFCPRRYKQIGTCGLPGTKCCKKP

SEQ ID NO: 9 - protein, 39 aa, Homo sapiens
QKYYCRVRGGRCAVLSCLPKEEQIGKCSTRGRKCCRRKK

SEQ ID NO: 10 - protein, 46 aa, Homo sapiens
LDRICGYGTARCRKKCRSQEYRIGRCPNTYACCLRKWDESLLNRTK

SEQ ID NO: 11 - protein, 103 aa, Homo sapiens
MRQRLLPSVTSLLLVALLFPGSSQARHVNHSATEALGELRERAPGQGTNGFQLLRHAVKRDLLPPRTPPYQVHISHREARGPSFRICVDFLGPRWARGCSTGN

SEQ ID NO: 12 - protein, 50 aa, Homo sapiens
MKVFFLFAVLFCLVQTNSVHISHREARGPSFRICVDFLGPRWARGCSTGN

SEQ ID NO: 13 - protein, 9 aa, Artificial
  FLAG tag
DYKDDDDKG

SEQ ID NO: 14 - DNA, 306 bp, Artificial
  variant SUMO
atgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtggtgctacgtattag

SEQ ID NO: 15 - protein, 101 aa, Artificial
  Synthetic Construct
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATY

SEQ ID NO: 16 - DNA, 33 bp, Artificial
  primer
gcgcgggcatatgggacttctccgcaaaggtgg

SEQ ID NO: 17 - DNA, 78 bp, Artificial
  primer
gcgcgctcgagctagcacttgtcgtcgtcgtccttgtagtctttttcttcttcttcctgctccggctgaggtacaagt

SEQ ID NO: 18 - DNA, 35 bp, Artificial
  primer
gcgcgctcgagctactgctccggctgaggtacaag

SEQ ID NO: 19 - DNA, 42 bp, Artificial
  primer
gcgcgccatatgggacttctccgcgaaggtggggaggagatt

SEQ ID NO: 20 - DNA, 75 bp, Artificial
  primer
ctactgctccggctgaggtacaagttcctgaaaaaaattctcaatttcctggccaatttcctcaagctcttcacc

SEQ ID NO: 21 - protein, 34 aa, Artificial
  negative control sequence
GLLREGGEEIGEELEEIGQEIENFFQELVPQPEQ

SEQ ID NO: 22 - protein, 134 aa, Artificial
  Exemplary SUMO-AMP fusion protein
SDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATYGLLRKGGEKIGEKLKKIGQKIKNFFQKLVPQPEQ

SEQ ID NO: 23 - protein, 135 aa, Artificial
  exemplary SUMO-AMP fusion protein
MGLLRKGGEKIGEKLKKIGQKIKNFFQKLVPQPEQSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATY

SEQ ID NO: 24 - protein, 138 aa, Artificial
  exemplary SUMO-AMP fusion protein.
SDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATYGGHMGLLRKGGEKIGEKLKKIGQKIKNFFQKLVPQPEQ

SEQ ID NO: 25 - protein, 142 aa, Artificial
  exemplary SUMO-AMP fusion protein.
MGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATYGLLRKGGEKIGEKLKKIGQKIKNFFQKLVPQPEQHHHHHH

SEQ ID NO: 26 - protein, 140 aa, Artificial
  exemplary SUMO-AMP fusion protein.
SDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATYHHHHHHGLLRKGGEKIGEKLKKIGQKIKNFFQKLVPQPEQ

SEQ ID NO: 27 - protein, 142 aa, Artificial
  Exemplary SUMO-AMP fusion protein
MGHHHHHHGLLRKGGEKIGEKLKKIGQKIKNFFQKLVPQPEQSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATY

SEQ ID NO: 28 - protein, 142 aa, Artificial; identical to SEQ ID NO: 27
  exemplary SUMO-AMP fusion protein.

SEQ ID NO: 29 - protein, 146 aa, Artificial
  exemplary SUMO-AMP fusion protein
MGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATYGGHMGLLRKGGEKIGEKLKKIGQKIKNFFQKLVPQPEQHHHHHH

SEQ ID NO: 30 - protein, 146 aa, Artificial
  Exemplary SUMO-AMP fusion protein
MGHHHHHHVVHMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATYGLLRKGGEKIGEKLKKIGQKIKNFFQKLVPQPEQ

SEQ ID NO: 31 - protein, 146 aa, Artificial
  Exemplary SUMO-AMP fusion protein
MGHHHHHHVVHMGLLRKGGEKIGEKLKKIGQKIKNFFQKLVPQPEQSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATY

SEQ ID NO: 32 - protein, 146 aa, Artificial
  exemplary SUMO-AMP fusion protein
MGHHHHHHSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATYGGHMGLLRKGGEKIGEKLKKIGQKIKNFFQKLVPQPEQ

SEQ ID NO: 33 - DNA, 38 bp, Artificial
  primer to amplify human LL37
gcgcaattccatatgaagacccaaagggatggccactc

SEQ ID NO: 34 - DNA, 36 bp, Artificial
  primer to amplify human LL37
gcgcctcgagctaggactctgtcctgggtacaagat

SEQ ID NO: 35 - DNA, 35 bp, Artificial
  primer to amplify human LL37
gcgcgccatatgctgctgggtgatttcttccggaa

SEQ ID NO: 36 - DNA, 513 bp, Homo sapiens
  CDS (1)..(513)
atgaagacccaaagggatggccactccctggggcggtggtcactggtgctcctgctgctgggcctggtgatgcctctggccatcattgcccaggtcctcagctacaaggaagctgtgcttcgtgctatagatggcatcaaccagcggtcctcggatgctaacctctaccgcctcctggacctggaccccaggcccacgatggatggggacccagacacgccaaagcctgtgagcttcacagtgaaggagacagtgtgccccaggacgacacagcagtcaccagaggattgtgacttcaagaaggacgggctggtgaagcggtgtatggggacagtgaccctcaaccaggccaggggctcctttgacatcagttgtgataaggataacaagagatttgccctgctgggtgatttcttccggaaatctaaagagaagattggcaaagagtttaaaagaattgtccagagaatcaaggattttttgcggaatcttgtacccaggacagagtcctag

SEQ ID NO: 37 - protein, 170 aa, Homo sapiens
MKTQRDGHSLGRWSLVLLLLGLVMPLAIIAQVLSYKEAVLRAIDGINQRSSDANLYRLLDLDPRPTMDGDPDTPKPVSFTVKETVCPRTTQQSPEDCDFKKDGLVKRCMGTVTLNQARGSFDISCDKDNKRFALLGDFFRKSKEKIGKEFKRIVQRIKDFLRNLVPRTES

SEQ ID NO: 38 - DNA, 114 bp, Homo sapiens
  CDS (1)..(114)
gccctgctgggtgatttcttccggaaatctaaagagaagattggcaaagagtttaaaagaattgtccagagaatcaaggattttttgcggaatcttgtacccaggacagagtcc

SEQ ID NO: 39 - protein, 38 aa, Homo sapiens
ALLGDFFRKSKEKIGKEFKRIVQRIKDFLRNLVPRTES

SEQ ID NO: 40 - protein, 149 aa, Artificial
  His tag-SUMO-LL37 fusion protein
MGHHHHHHSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATYGGHMLLGDFFRKSKEKIGKEFKRIVQRIKDFLRNLVPRTES

SEQ ID NO: 41 - protein, 34 aa, Artificial
  mutant CRAMP peptide
DFFRKSKEKIGEKLKKIGQKIKNFFQKLVPQPEQ

SEQ ID NO: 42 - DNA, 42 bp, Artificial
  primer to generate mutated CRAMP
gcgcgccatatggatttcttccgcaaatctaaagagaagatt

SEQ ID NO: 43 - protein, 34 aa, Artificial
  mutant CRAMP peptide
GLLRKGGEKIGEKLKKIGQKIKNFFQNLVPRTES

SEQ ID NO: 44 - DNA, 37 bp, Artificial
  primer
gcgcctcgagctaggactctgtccggggtacaagatt

SEQ ID NO: 45 - protein, 155 aa, Artificial
  His-SUMO-CRAMP-FLAG tag fusion protein
MGHHHHHHSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATYGGHMGLLRKGGEKIGEKLKKIGQKIKNFFQKLVPQPEQDYKDDDDKG

SEQ ID NO: 46 - protein, 45 aa, Artificial
  Resulting peptide when SEQ ID NO 45 treated with sumoase
HMGLLRKGGEKIGEKLKKIGQKIKNFFQKLVPQPEQDYKDDDDKG

SEQ ID NO: 47 - protein, 146 aa, Artificial; identical to SEQ ID NO: 32
  His-SUMO-CRAMP fusion protein

SEQ ID NO: 48 - protein, 36 aa, Artificial
  resulting protein when SEQ ID NO 47 treated with sumoase
HMGLLRKGGEKIGEKLKKIGQKIKNFFQKLVPQPEQ

SEQ ID NO: 49 - protein, 146 aa, Artificial
  His-SUMO-CRAMP-M1 fusion protein
MGHHHHHHSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATYGGHMDFFRKSKEKIGEKLKKIGQKIKNFFQKLVPQPEQ

SEQ ID NO: 50 - protein, 36 aa, Artificial
  resulting peptide when SEQ ID NO 40 treated with sumoase
HMDFFRKSKEKIGEKLKKIGQKIKNFFQKLVPQPEQ

SEQ ID NO: 51 - protein, 146 aa, Artificial
  His-SUMO-CRAMP-M2 fusion protein
MGHHHHHHSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGATYGGHMGLLRKGGEKIGEKLKKIGQKIKNFFQNLVPRTES

SEQ ID NO: 52 - protein, 36 aa, Artificial
  resulting sequence when SEQ ID NO 51 treated with sumoase.
HMGLLRKGGEKIGEKLKKIGQKIKNFFQNLVPRTES

SEQ ID NO: 53 - protein, 39 aa, Artificial
  resulting peptide when SEQ ID NO 40 treated with sumoase.
HMLLGDFFRKSKEKIGKEFKRIVQRIKDFLRNLVPRTES

(19) United States
(12) Patent Application Publication
(10) Pub. No.: US 2010/0048480 A1
(43) Pub. Date: Feb. 25, 2010

(54) PRODUCTION OF ANTI-MICROBIAL PEPTIDES

(76) Inventors: Bettina Bommarius, Atlanta, GA (US); Melanie Sherman, Stone Mountain, GA (US); Daniel Kalman, Atlanta, GA (US); Xiadong Cheng, Decatur, GA (US)

Correspondence Address: KLARQUIST SPARKMAN, LLP, 121 SW SALMON STREET, SUITE 1600, PORTLAND, OR, 97204, US

(21) Appl. No.: 12/515,530

(22) PCT Filed: Nov. 20, 2007
(86) PCT No.: PCT/US07/85288

§ 371 Date: May 19, 2009

Related U.S. Application Data
(60) Provisional application No. 60/867,070, filed on Nov. 22, 2006.

(51) Int. Cl. A61K 38/16; C07K 14/00; C07H 21/04; C12N 15/63; C12N 5/00; C12P 21/06

(52) U.S. Cl. 514/12; 530/350; 536/23.4; 435/320.1; 435/325; 435/69.7

(57) ABSTRACT

The present application provides methods of producing antimicrobial peptides (AMPs) in a cell, for example by expression a fusion protein that includes small ubiquitin related modifier (SUMO) and an AMP in the cell. Also provided are nucleic acid and protein sequences of SUMO-AMP fusion proteins, and kits that include such molecules.

### CROSS REFERENCE TO RELATED APPLICATIONS

This application claims priority to, and the benefit of, U.S. Provisional Patent Application No. 60/867,070, filed on Nov. 22, 2006, which is incorporated herein in its entirety.

### FIELD

This application relates to methods of producing anti-microbial peptides, for example by expressing a small ubiquitin related modifier (SUMO)-antimicrobial peptide fusion protein in a cell, SUMO-antimicrobial peptide fusion proteins and nucleic acid molecules that encode such proteins, and well as kits that include such proteins.

### BACKGROUND

Antimicrobial peptides (AMPs) are part of the innate immunity defense line in mammals and other organisms (such as plants and invertebrates) and generally function to destroy microbes, such as bacteria, for example by generating holes into the membrane of the microbe. The inherent antimicrobial activity of AMPs makes recombinant expression of these peptides in a bacterial cell difficult. For example, recombinant expression of AMP in a bacterial cell often kills the cells as the AMP is produced. Because AMPs are about 12-100 amino acids in length, synthesizing these peptides can be expensive, especially if large quantities are need (for example for therapeutic purposes). Therefore, methods are needed that can be used to express AMPs without significantly killing the cells in which the AMP is expressed.

### SUMMARY

The inventors have determined that recombinant expression of antimicrobial peptides (AMPs) in a cell can be achieved by expressing the AMP as a fusion protein. Expression of an AMP fusion protein, for example by attaching a SUMO sequence to the N-terminus of a CRAMP AMP or LL37 AMP, permitted high levels of SUMO-CRAMP or SUMO-LL37 fusion protein expression in bacterial cells, respectively. The SUMO sequence can be cleaved (for example after protein purification) using a protease (sumoase), thereby releasing biologically active AMP (such as CRAMP or LL37).

Based on these observations, provided by this disclosure are methods of producing a protein, such as a SUMO-AMP fusion protein. In particular examples the method includes expressing a nucleic acid molecule encoding the fusion protein in a recombinant or transgenic cell, such as a nucleic acid molecule encoding SUMO (or other ubiquitin like protein) operably linked (and in frame to) to a nucleic acid molecule encoding an AMP. The cell is cultured under conditions sufficient for expression of the fusion protein. Linkage of a SUMO sequence to an AMP sequence can significantly decrease the native antimicrobial activity of the AMP (for example thereby significantly reducing or inhibiting the ability of the AMP to kill the host cell in which it is expressed), increase the stability of the fusion protein (for example such that the AMP degrades more slowly than in the absence of the SUMO sequence), or combinations thereof. In particular examples, the method further includes purifying the SUMO-AMP fusion protein. In some examples, method further includes removing SUMO from the fusion protein, thereby restoring antimicrobial activity to the AMP. In particular examples, this expression system for AMPs is capable of producing high amounts of AMP (such as at least 2500 μg/L of bacterial culture, for example at least 3000 μg/L, at least 4000 μg/L, such as 3000-5000 μg/L of bacterial culture). In some examples, the disclosed methods permit generation of commercial amounts of AMPs without the use of cost-intensive peptide synthesis.

Also provided are purified fusion peptides produced using the methods provided herein, such as SUMO-AMP proteins. Non-limiting examples are provided in SEQ ID NOS: 22-32, 40, 45, 47, 49 and 51. Isolated nucleic acid molecules that encode the disclosed fusion proteins are provided, as well as vectors and cells that include such nucleic acid molecules. Herein disclosed are kits that include SUMO-AMP proteins, or nucleic acids encoding such proteins (such vectors), as well as a sumoase, cells suitable for expression of a vector, lysis buffer, wash buffer, elution buffer, or combinations thereof.

Methods are provided for treating an infection in a subject, such as treating a disease that results from bacterial fungal, parasitic, or viral infection. In particular examples, the method includes administering to the subject a therapeutically effective amount of one or more AMPs produced by the methods provided herein, thereby treating the infection (for example by relieving one or more symptoms associated with the infection).

The foregoing and other objects and features of the disclosure will become more apparent from the following detailed description, which proceeds with reference to the accompanying figures.

### BRIEF DESCRIPTION OF THE DRAWINGS

FIG. 1 is a digital image of a protein gel showing SUMO-CRAMP purification and proteolytic digest of the fusion protein with sumoase. Lane 1 shows the purified sumoase (28 kD) and lane 2 shows the fusion protein SUMO-CRAMP (14.4 kD), the cleaved SUMO protein (11 kD) as well as the cleaved CRAMP product (3.74 kD).

FIG. 2 is a bar graph showing the functionality of BBcramp in comparison to commercially available CRAMP.

FIG. 3 is a bar graph showing the functionality of CRAMP (native and two mutants) and LL37 generated using the methods provided herein in comparison to commercially available LL37 (pos LL37).

FIG. 4 is a digital image of a 16% tricine gel of the equally expressed and partially purified antimicrobial peptides (CRAMP and LL37), arrows indicate the released active peptide after sumoase treatment of the SUMO-AMP expressed fusion proteins.

### SEQUENCE LISTING

The nucleic and amino acid sequences listed in the accompanying sequence listing are shown using standard letter abbreviations for nucleotide bases, and three letter code for amino acids. Only one strand of each nucleic acid sequence is shown, but the complementary strand is understood as included by any reference to the displayed strand.

SEQ ID NOS: 1 and 2 show exemplary cathelicidin protein sequences (LL37 and CRAMP, respectively).

SEQ ID NOS: 3-6 show exemplary α-defensin protein sequences (HNP-1, HNP-4, HD-5, and HD-6, respectively).

SEQ ID NOS: 7-10 show exemplary β-defensin protein sequences (HBD-1, HBD-2, HBD-3, and HBD-4, respectively).

SEQ ID NOS: 11-12 show exemplary human epididymis 2 (EP2) protein sequences He-2α and HE-2β1, respectively), wherein the active fragment can include amino acids 72-103 of SEQ ID NO: 11 or amino acids 19-50 of SEQ ID NO: 12.

SEQ ID NO: 13 shows an amino acid sequence of a FLAG purification tag.

SEQ ID NOS: 14-15 show an exemplary SUMO nucleic acid coding sequence, and the resulting protein sequence, respectively. This SUMO sequence is a variant of the wild-type yeast SUMO sequence.

SEQ ID NOS: 16-20 are primers used to amplify the full-length gene of CRAMP and to insert a FLAG tag on the C-terminus of CRAMP.

SEQ ID NO: 21 shows an exemplary negative control protein sequence, pramc.

SEQ ID NOS: 22-32 show exemplary SUMO-AMP fusion proteins.

SEQ ID NOS: 33-35 are primers used to amplify a full-length human LL37.

SEQ ID NOS: 36 and 37 show an exemplary human LL37 cDNA and protein sequence, respectively.

SEQ ID NOS: 38 and 39 show an exemplary active portion of human LL37 cDNA and protein, respectively.

SEQ ID NO: 40 shows an exemplary HIS tag-SUMO-LL37 fusion protein sequence.

SEQ ID NO: 41 shows a mutated CRAMP protein sequence (CRAMP-M1).

SEQ ID NO: 42 is a primer used to generate SEQ ID NO: 41.

SEQ ID NO: 43 shows a mutated CRAMP protein sequence (CRAMP-M2).

SEQ ID NO: 44 is a primer used to generate SEQ ID NO: 43.

SEQ ID NO: 45 shows an exemplary HIS tag-SUMO-CRAMP-FLAG-tag fusion protein sequence.

SEQ ID NO: 46 shows the resulting protein (CRAMP-FLAG-tag) when SEQ ID NO: 45 is treated with sumoase.

SEQ ID NO: 47 shows an exemplary HIS tag-SUMO-CRAMP fusion protein sequence.

SEQ ID NO: 48 shows the resulting protein (CRAMP) when SEQ ID NO: 47 is treated with sumoase.

SEQ ID NO: 49 shows an exemplary HIS tag-SUMO-CRAMP-M1 fusion protein sequence.

SEQ ID NO: 50 shows the resulting protein (CRAMP-M1) when SEQ ID NO: 49 is treated with sumoase.

SEQ ID NO: 51 shows an exemplary HIS tag-SUMO-CRAMP-M2 fusion protein sequence.

SEQ ID NO: 52 shows the resulting protein (CRAMP-M2) when SEQ ID NO: 51 is treated with sumoase.

SEQ ID NO: 53 shows the resulting protein (LL37) when SEQ ID NO: 40 is treated with sumoase.

### DETAILED DESCRIPTION

### Abbreviations and Terms

The following explanations of terms and methods are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. For example, reference to “comprising an antimicrobial peptide” includes one or a plurality of such antimicrobial peptides, and reference to “comprising the fusion protein” includes reference to one or more test fusion proteins and equivalents thereof known to those skilled in the art, and so forth. The term “or” refers to a single element of stated alternative elements or a combination of two or more elements, unless the context clearly indicates otherwise. For example, the phrase “A or B” refers to A, B, or a combination of both A and B. Reference to GenBank Accession Nos. include sequences publicly available as of Nov. 22, 2006.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.
AMP: antimicrobial peptide
SUMO: small ubiquitin related modifier

Administration: To provide or give a subject an agent, such as an AMP produced using the methods provided herein, by any effective route. Exemplary routes of administration include, but are not limited to, oral, injection (such as subcutaneous, intramuscular, intradermal, intraperitoneal, and intravenous), sublingual, rectal, transdermal, intranasal, vaginal and inhalation routes.

Antimicrobial peptide (AMP): Proteins involved in the innate immunity of numerous organisms, including bacteria, fungi, plants, invertebrates, and vertebrates. Referred to as cationic peptides by some in the art. Initially it was proposed that these peptides had antimicrobial activity. However, recent evidence indicates that AMPs have a diverse range of functions in modulating immunity, which have an impact on infection and inflammation.

AMPs exhibit broad-spectrum activity against Gram-positive and Gram-negative bacteria, yeasts, fungi, parasites, and enveloped viruses. Typically, AMPs are short, such as about 12-100 amino acids (for example 12-50 amino acids), are positively charged (such as net charge of about +2 to +9), are amphiphilic. In particular examples, AMPs include 2-9 positively charged lysine or arginine residues, and up to 50% hydrophobic amino acids. To date, hundreds of AMPs are recognized by those skilled in the art. Despite their similar generally physical properties, individual AMPs have very limited sequence homologies and a wide range of secondary structures.

Particular examples of AMPs include, but are not limited to, cathelicidins, defensins, human epididymis (HE2) peptides, histatins, magainins, mellitins, temporins, dermaseptins, cecropins, as well as numerous others known in the art (for example see Jenssen et al., Clin. Microl. Rev. 19:491-511, 2006 for a review article).

Antimicrobial activity: The ability of one or more agents, such as an AMP, to retard or decrease the growth of one or more microbes, and in some examples inhibit the growth of or even kill one or more microbes, such as those microorganisms that cause disease (for example bacteria, viruses, fungi, and parasites). Includes antibacterial, antifungal, antiparasitic, and antiviral activities. In a specific example, an anti-microbial agent can reduce the growth of or kill at least 90% of microbes present, such as least 99%. Examples of measuring antimicrobial activity are known in the art (Example 3 provides an exemplary method).

cDNA (complementary DNA): A piece of DNA lacking internal, non-coding segments (introns) and regulatory sequences which determine transcription. cDNA can be synthesized by reverse transcription from messenger RNA extracted from cells.

Cathelicidin: A family of AMPs that contain a highly conserved signal sequence and a proregion highly homologous to cathelin and variable C-terminal sequences that correspond to the mature AMP. About 30 members of this family have been identified. Examples include LL37 (e.g., SEQ ID NO: 1, 39, or amino acids 2-37 of SEQ ID NO: 1), CRAMP (e.g., SEQ ID NO: 2, 41, and 43), CRAMP-18 (amino acids 10-28 of SEQ ID NO: 2), CAP18, PMAP-36, BMAP-28, protegrins, bactenecin 5, and bacterecin 7.

Cathelicidin sequences are publicly available. For example, GenBank Accession Nos: NP_{—}004336 (the 37 terminal amino acids) and NM_{—}004345 disclose exemplary human LL37 peptide and nucleic acid sequences, respectively and GenBank Accession Nos: NP_{—}034051 (amino acids 150-167) and NM_{—}009921 disclose exemplary CRAMP-18 peptide and nucleic acid sequences, respectively.

However, one skilled in the art will appreciate that cathelicidin variants (such as fragments, or sequences containing substitutions, deletions, or insertions, or combinations thereof), that retain cathelicidin biological activity, can be used. For example, a cathelicidin can have at least 80% sequence identity, for example at least 85%, at least 90%, at least 95%, or at least 98% sequence identity to any publicly available cathelicidin sequence. Exemplary fragments of LL37 that can be used in a SUMO-AMP fusion protein include amino acids 15-32 of SEQ ID NO: 1 and amino acids 2-37 of SEQ ID NO: 1. Exemplary variant LL37 sequences that can be used in a SUMO-AMP fusion protein include replacement of E¹⁶ and K²⁵ with Leu (L), replacement of A²², D²⁶, and N³⁰ with Lys (K), or combinations thereof (either in the full-length LL37 sequence or a fragment thereof such as amino acids 2-37 or 15-32 of SEQ ID NO: 1). An exemplary fragment of CRAMP that can be used in a SUMO-AMP fusion protein is amino acids 10-28 of SEQ ID NO: 2. Exemplary variant CRAMP or CRAMP-18 sequences that can be used in a SUMO-AMP fusion protein include replacement of residues 2, 9, 13, 16 (or combinations thereof) with Lys (K), or combinations thereof (amino acids refer to amino acids 10-28 SEQ ID NO: 2). Specific exemplary variant CRAMP sequences are shown in SEQ ID NOS: 41 and 43. In some examples, such substitutions enhance the antimicrobial activity of the peptide.

Conservative substitution: One or more amino acid substitutions (for example 1, 2, 5 or 10 amino acid residues) for amino acid residues having similar biochemical properties. Typically, conservative substitutions have little to no impact on the activity of a resulting peptide. For example, a conservative substitution is an amino acid substitution in an AMP that does not substantially decrease the antimicrobial activity of the peptide. In another example, a conservative substitution is an amino acid substitution in a SUMO peptide that does not significantly alter the ability of the SUMO, in the presence of a SUMO-AMP fusion protein, to decrease the antimicrobial activity of the AMP. For example 1 or more conservative substitutions (such as 1, 2, 5, 10, or 12 conservative substitutions) can be made to the SUMO-AMP fusion proteins shown in SEQ ID NOS: 22-32, 40, 45, 47, 49 and 51.

An alanine scan can be used to identify amino acid residues in a peptide that can tolerate substitution. In one example, the antimicrobial activity of an AMP is not decreased by more than 25%, for example not more than 20%, for example not more than 10%, when an alanine, or other conservative amino acid (such as those listed below), is substituted for one or more native amino acids in the AMP.

A peptide can be produced to contain one or more conservative substitutions by manipulating the nucleotide sequence that encodes that peptide using, for example, standard procedures such as site-directed mutagenesis or PCR. Alternatively, a peptide can be produced to contain one or more conservative substitutions by using standard peptide synthesis methods.

Substitutional variants are those in which at least one residue in the amino acid sequence has been removed and a different residue inserted in its place. Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

Further information about conservative substitutions can be found in, among other locations in, Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988), WO 00/67796 (Curd et al.) and in standard textbooks of genetics and molecular biology.

Culture: To incubate microorganisms or cells in media that permits their growth. For example, culturing cells in media can be used to permit expression of recombinant nucleic acid molecules encoding the SUMO-AMP fusion proteins disclosed herein.

Decrease: To reduce the quality, amount, or strength of something.

In one example, the presence of SUMO in a fusion protein that includes an AMP significantly decreases the biological activity of the AMP. For example, the antimicrobial activity of a SUMO-AMP fusion protein is significantly decreased relative to the AMP protein alone. In a particular example, the antimicrobial activity of a SUMO-AMP fusion protein is decreased by at least 25%, at least 50%, at least 80%, at least 90%, or even at least 99%, relative to the AMP protein alone. Such decreases can be measured using the methods disclosed herein (for example, see Example 3).

Defensin: A cationic non-glycosylated antimicrobial peptide that contains six cysteine residues that form three intramolecular disulfide bridges, resulting in a triple-stranded β-sheet structure. Defensins are usually 3.5-6 kDa, and their amphipathic structure permits insertion into microbial membranes, resulting in permeabilization of the plasma membrane (and killing the microbe). In humans, there are two classes of defensins: α- and β-defensins. Sequences of defensins are publicly available (for example on GenBank and EMBL).

α-defensins are generally 29-35 amino acids in length, with three disulfide bridges between residues 1 and 6, 2 and 4, and 3 and 5. Particular non-limiting examples of α-defensins include HNP-1 (SEQ ID NO: 3), HNP-4 (SEQ ID NO: 4), HD-5 (SEQ ID NO: 5), and HD-6 (SEQ ID NO: 6).

β-defensins are generally larger than α-defensins, with three disulfide bridges between residues 1 and 5, 2 and 4, and 3 and 6. Particular non-limiting examples of β-defensins include HBD-1 (SEQ ID NO: 7), HBD-2 (SEQ ID NO: 8), HBD-3 (SEQ ID NO: 9) and HBD-4 (SEQ ID NO: 10).

Defensin sequences are publicly available, for example from GenBank or EMBL (see SEQ ID NOS: 4-10). However, one skilled in the art will appreciate that defensin variants (such as fragments, or sequences containing substitutions, deletions, or insertions, or combinations thereof), that retain defensin biological activity, can be used. For example, a defensin can have at least 80% sequence identity, for example at least 85%, at least 90%, at least 95%, or at least 98% sequence identity to any publicly available defensin sequence. In some examples, such variations enhance the antimicrobial activity of the defensin peptide. An exemplary fragment of HBD2 that can be used in a SUMO-AMP fusion protein is amino acids 2-40 of SEQ ID NO: 8.

DNA (Deoxyribonucleic acid): A long chain polymer which includes the genetic material of most living organisms (some viruses have genes including ribonucleic acid, RNA). The repeating units in DNA polymers are four different nucleotides, each of which includes one of the four bases, adenine, guanine, cytosine and thymine bound to a deoxyribose sugar to which a phosphate group is attached. Triplets of nucleotides, referred to as codons, in DNA molecules code for amino acid in a peptide. The term codon is also used for the corresponding (and complementary) sequences of three nucleotides in the mRNA into which the DNA sequence is transcribed.

Encode: When one piece of information (for example, a letter, word, or phrase) represents another form or representation. For example, a nucleic acid molecule that provides the desired protein sequence when translated, is said to “encode” the desired protein sequence (such as a SUMO-AMP sequence). Three consecutive nucleotides in a DNA molecule dictate the particular amino acid inserted into the resulting protein molecule. Due to the degeneracy of the genetic code, several different nucleic acid molecules can encode for the same protein. For example, the codons GCA, GCG, GCT, and GCC all encode for alanine.

Enhance or increase: To improve the quality, amount, or strength of something.

In one example, the presence of SUMO in a fusion protein that includes an AMP enhances the stability of the fusion protein or AMP. For example, the stability of a SUMO-AMP fusion protein is enhanced relative to the AMP protein alone. In one example, enhancing the stability of a protein protects the protein from misfolding or unfolding. In a particular example, the stability of a SUMO-AMP fusion protein is increased by at least 25%, at least 50%, at least 80%, at least 90%, or even at least 99%, relative to the AMP protein alone. Such stability (for example protection from unfolding) can be measured using methods known in the art, for example using circular dichroism to measure the a-helix content of the peptide, wherein the active peptide is an amphipathic a-helix (for example see Matsuo et al., J. Biochem. 135:405-11, 2004).

Exogenous: The term “exogenous” as used herein with reference to a nucleic acid molecule and a particular cell refers to any nucleic acid molecule that does not originate from that particular cell as found in nature, such as a nucleic acid molecule that encodes a SUMO-AMP. Thus, a non-naturally-occurring nucleic acid molecule is considered to be exogenous to a cell once introduced into the cell. A nucleic acid molecule that is naturally-occurring also can be exogenous to a particular cell. For example, an entire coding sequence isolated from cell X is an exogenous nucleic acid with respect to cell Y once that coding sequence is introduced into cell Y, even if X and Y are the same cell type.

Expression: The process by which a nucleic acid molecule's coded information is converted into a protein, transfer RNA, or ribosomal RNA. Expressed nucleic acid molecule's include those that are transcribed into mRNA and then translated into protein and those that are transcribed into RNA but not translated into protein (for example, transfer and ribosomal RNAs).

Fusion protein: A protein that includes two or more protein sequences that are not linked together in nature. Fusion proteins can be generated using recombinant molecular biology methods. For example, a fusion gene can be created by operably linking two or more protein coding sequences in frame. The resulting DNA sequence will then be expressed by a cell as a single protein. The DNA sequence can be part of a vector, wherein the vector is introduced into eukaryotic or prokaryotic cells. Once inside the cell the vector allows the protein to be produced.

In one example, a fusion protein includes SUMO (or other ubiquitin-like sequence) attached to an AMP (referred to herein as SUMO-AMP). SUMO can be attached to the N- or C-terminus of AMP (therefore the term SUMO-AMP does not designate the particular termini to which SUMO is attached). In some examples, SUMO is directly attached to AMP. In other examples, a linker (or “spacer”) separates SUMO from the AMP, such as a linker of at least 1 amino acid, such as at least 4, or at least 10 amino acids, for example, 2, 4, 5, 8, 9, 10, 12, 15, 20, 30, or 50 amino acid residues. In some examples, a SUMO-AMP fusion protein also includes a purification tag. Exemplary SUMO-AMP fusion proteins are shown in SEQ ID NOS: 22-32, 40, 45, 47, 49 and 51.

Vectors that can be used to produce SUMO fusion proteins are publicly available (for example The Champion™ pET SUMO Protein and Peptide Expression System from Invitrogen can be used to produce SUMO-AMP fusion proteins in E. coli). After expression of the SUMO-AMP protein, the 11 kd SUMO moiety can be cleaved using a sumoase, producing a functional AMP.

Human epididymis 2 (EP2): A gene expressed in human epididymis that encodes a family of small cationic secretory AMPs of 4-8 kDa. Particular examples include He-2α (SEQ ID NO: 11) and HE-2β1 (SEQ ID NO: 12), as well as the active fragment thereof (such as amino acids 72-103 of SEQ ID NO: 11 and amino acids 19-50 of SEQ ID NO: 12).

EP2 sequences are publicly available, for example from GenBank or EMBL (see SEQ ID NOS: 11 and 12). However, one skilled in the art will appreciate that EP2 variants (such as fragments, or sequences containing substitutions, deletions, or insertions, or combinations thereof), that retain EP2 biological activity, can be used. For example, an EP2 can have at least 80% sequence identity, for example at least 85%, at least 90%, at least 95%, or at least 98% sequence identity to any publicly available EP2 sequence (such as that shown in amino acids 72-103 of SEQ ID NO: 11). In some examples, such variations enhance the antimicrobial activity of the EP2 peptide.

Isolated: An “isolated” biological component (such as a protein or nucleic acid molecule) has been substantially separated or purified away from other biological components of the cell or organism in which the component naturally occurs.

For example, an isolated fusion protein or AMP is one in which the protein has been substantially separated or purified away from other cellular components in which the peptide was produced. For example, an isolated fusion protein or AMP is substantially separated or purified away from other proteins and other cellular components such as nucleic acid molecules.

Linker: A structure that joins one molecule to another, such as attaches one protein (such as SUMO) to another protein (such as an AMP), for example to form a SUMO-AMP fusion peptide disclosed herein. For example, one portion of the linker can be operably linked to SUMO, and another portion of the linker operably linked to AMP.

Exemplary linkers include amino acids, such as alanine or glycine. Linkers can be of any length, such as 1-50, 1-5, or 1-2 amino acids.

Nucleic acid molecule: A deoxyribonucleotide or ribonucleotide polymer in either single or double stranded form, and unless otherwise limited, encompasses known analogues of natural nucleotides that hybridize to nucleic acids in a manner similar to naturally occurring nucleotides.

ORF (open reading frame): A series of nucleotide triplets (codons) coding for amino acids without any termination codons. These sequences are usually translatable into a peptide.

Operably linked: A first nucleic acid sequence (such as a sequence encoding SUMO) is operably linked with a second nucleic acid sequence (such as a sequence encoding an AMP) when the first nucleic acid sequence is placed in a functional relationship with the second nucleic acid sequence. For instance, a promoter is operably linked to a coding sequence if the promoter affects the transcription or expression of the coding sequence. In another example, a SUMO sequence is operably linked to an AMP sequence if the expression of the nucleic acid sequence results in production of a SUMO-AMP fusion protein. Generally, operably linked DNA sequences are contiguous and, where necessary to join two protein coding regions (for example to generate a fusion protein), in the same reading frame.

Promoter: An array of nucleic acid control sequences that directs transcription of a nucleic acid molecule, such as a nucleic acid molecule encoding a fusion protein. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. The term includes endogenous promoter sequences as well as exogenous promoter sequences.

Particular types of promoters that can be used to practice the methods disclosed herein include, but are not limited to, constitutive promoters and inducible promoters (such as a promoter responsive or unresponsive to a particular stimulus, for example such as light, oxygen, or chemical concentration, such as a lactose, IPTG, or tetracycline). Specific, non-limiting examples of promoters include promoters derived from the genome of mammalian cells (such as a metallothionein promoter) or from mammalian viruses (such as the retrovirus long terminal repeat; the adenovirus late promoter; or the vaccinia virus 7.5K promoter) can be used. Promoters produced by recombinant DNA or synthetic techniques can also be used.

A nucleic acid sequence encoding a SUMO-AMP fusion protein can be inserted into an expression vector that contains a promoter sequence which facilitates the efficient transcription of the SUMO-AMP fusion protein. The expression vector typically contains an origin of replication, a promoter, and can include specific nucleic acid sequences (such as an antibiotic resistance marker) that allow phenotypic selection of the transformed cells.

Protease: An enzyme that breaks peptide bonds between amino acids of proteins. This process, often referred to a peptide cleavage, can be used to activate or inactivate a protein. For example, the action of a protease on a SUMO-AMP fusion protein can liberate AMP from SUMO, thereby restoring antimicrobial activity to AMP. An exemplary protease is sumoase.

Purified: The term purified does not require absolute purity; rather, it is intended as a relative term. Thus, for example, a purified peptide preparation is one in which the peptide is more enriched than the peptide is in its environment within a cell or extracellular medium, such that the peptide is substantially separated from cellular components (nucleic acids, lipids, carbohydrates, and other polypeptides) or components of an extracellular medium that may accompany it. In some examples, a purified peptide preparation is one in which the peptide is substantially-free from contaminants, such as those that might be present following chemical synthesis of the peptide.

In one example, a SUMO-AMP fusion peptide or an AMP is purified when at least 60% by weight of a sample is composed of the peptide, for example when 75%, 95%, or 99% or more of a sample is composed of the peptide. Examples of methods that can be used to purify a protein, include, but are not limited to the methods disclosed in Sambrook et al. (Molecular Cloning: A Laboratory Manual, Cold Spring Harbor, N.Y., 1989, Ch. 17). Protein purity can be determined by, for example, polyacrylamide gel electrophoresis of a protein sample, followed by visualization of a single polypeptide band upon staining the polyacrylamide gel; high-pressure liquid chromatography; sequencing; or other conventional methods.

Purification tag: A sequence of amino acids that can be attached to a protein (such as a fusion protein) to permit purification of the protein from a cell or extracellular medium, for example using affinity chromatography. Also referred to in the art as an affinity tag. Particular non-limiting examples include glutathione-S-transferase (GST) protein (a 220 amino acid tag), FLAG peptide (such as that shown in SEQ ID NO: 13), a polyhistidine tag (His-tag) such as a 6×His-tag, an HA-tag, a myc-tag, or combinations thereof. Such tags can be fused to the C-terminus or the N-terminus of a protein (or both ends).

Recombinant: A recombinant nucleic acid is one that has a sequence that is not naturally occurring or has a sequence that is made by an artificial combination of two otherwise separated segments of sequence. This artificial combination can be accomplished by methods known in the art, such as chemical synthesis or by the artificial manipulation of isolated segments of nucleic acids, such as by genetic engineering techniques. Similarly, a recombinant protein is one encoded for by a recombinant nucleic acid molecule. A recombinant cell includes at least one recombinant nucleic acid molecule or recombinant protein.

Remove: To take away. Therefore, can include taking away at least one amino acid from a protein, for example, by enzymatic digestion (such as a protease). In a particular example, includes taking away the SUMO portion of a SUMO-AMP fusion protein using sumoase, thereby leaving an intact AMP protein. Such removal can occur inside of a cell (in vivo) or external to a cell (in vitro).

Restore: To return to its original function. For example, the native antimicrobial activity of an AMP is “masked” or decreased if SUMO is attached to AMP (thereby generating a SUMO-AMP fusion protein), and this antimicrobial activity can be returned to AMP by removal of the SUMO sequence.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. To compare two nucleic acid sequences, the options can be set as follows: -i is set to a file containing the first nucleic acid sequence to be compared (such as C:\seq1.txt); -j is set to a file containing the second nucleic acid sequence to be compared (such as C:\seq2.txt); -p is set to blastn; -o is set to any desired file name (such as C:\output.txt); -q is set to −1; -r is set to 2; and all other options are left at their default setting. For example, the following command can be used to generate an output file containing a comparison between two sequences: C:\B12seq -i c:\seq1.txt -j c:\seq2.txt -p blastn -o c:\output.txt -q−1 -r2.

To compare two amino acid sequences, the options of B12seq can be set as follows: -i is set to a file containing the first amino acid sequence to be compared (such as C:\seq1.txt); -j is set to a file containing the second amino acid sequence to be compared (such as C:\seq2.txt); -p is set to blastp; -o is set to any desired file name (such as C:\output.txt); and all other options are left at their default setting.

For example, the following command can be used to generate an output file containing a comparison between two amino acid sequences: C:\B12seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (i.e., 15÷20*100=75).

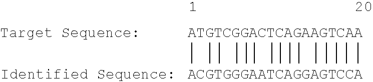

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least 75%, at least 80%, at least 85%, at least 90%, at least 95%, at least 97%, or at least 99% sequence identity to any known AMP or SUMO sequence (or any SUMO-AMP fusion protein sequence), such as at least this amount of sequence identity to any of SEQ ID NOS: 1-12, 15, 22-32, 37, 39-41, 43, and 45-53.

When aligning short peptides (fewer than around 30 amino acids), the alignment can be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least 60%, at least 70%, at least 75%, at least 80%, at least 85%, at least 90%, at least 95%, at least 98%, at least 99% sequence identity to any known AMP or SUMO sequence (or any SUMO-AMP fusion protein sequence), such as at least this amount of sequence identity to any of SEQ ID NOS: 1-12, 15, 22-32, 37, 39-41, 43 and 45-53. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence (such as an AMP or SUMO sequence). Methods for determining sequence identity over such short windows are described at the NCBI web site.

One indication that two nucleic acid molecules are closely related is that the two molecules hybridize to each other under stringent conditions. Stringent conditions are sequence-dependent and are different under different environmental parameters. For example, nucleic acid molecules that hybridize under stringent conditions to an AMP or SUMO (or a SUMO-AMP) coding sequence may also code for a functional AMP or SUMO (or a SUMO-AMP).

Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least 60%, at least 70%, at least 80%, at least 90%, at least 95%, at least 98%, or at least 99% sequence identity to any known AMP or SUMO coding sequence (or any SUMO-AMP fusion protein coding sequence), such as at least this amount of sequence identity to SEQ ID NO: 14 or any nucleic acid molecule that encodes any of SEQ ID NOS: 1-12, 15, 22-32, 37, 39-41, 43 and 45-53.

One of skill in the art will appreciate that these sequence identity ranges are provided for guidance only; it is possible that strongly significant homologs could be obtained that fall outside the ranges provided.

An alternative (and not necessarily cumulative) indication that two nucleic acid sequences are substantially identical is that the peptide which the first nucleic acid encodes is immunologically cross reactive with the peptide encoded by the second nucleic acid.

SUMO (small ubiquitin related modifier): A small ubiquitin-like protein that becomes covalently conjugated to a variety of target proteins, the large majority of which are found in the nucleus. SUMO is also referred to in the art as Sentrin, SMT3, PIC1, GMP1, and UBL1.

SUMO sequences are publicly available. For example, GenBank Accession Nos: NP_{—}003343 (human), ABB04499 (silkworm), and Q12306 (yeast) disclose exemplary SUMO protein sequences and GenBank Accession Nos: NM_{—}003352 (human), DQ192277 (silkworm), and NM_{—}019929 (mouse) disclose exemplary SUMO nucleic acid sequences. SUMO sequence have also been obtained from Arabidopsis sumo proteins (such as atsumo 1 and 2), which share about 51-75% identity with yeast smt3 and can be cleaved by Ulp1. Additional exemplary SUMO protein and nucleic acid sequences are provided in U.S. Pat. No. 7,060,461 (see SEQ ID NOS: 1, 2, 63 and 64 of U.S. Pat. No. 7,060,461, as well as SEQ ID NOS: 14 and 15 herein). However, one skilled in the art will appreciate that SUMO variants (such as fragments, sequences containing substitutions, deletions, or insertions, or combinations thereof), that retain SUMO activity, can be used. For example, SUMO can have at least 50% sequence identity, for example at least 60%, at least 75%, at least 85%, at least 90%, at least 95%, or at least 98% sequence identity to any publicly available SUMO sequence.

Sumoase (SUMO protease): A desumoylating enzyme. Therefore, a sumoase can be used to cleave SUMO from a SUMO-AMP fusion protein.

Examples include the Ubl-specific protease (Ulp)1 and Ulp2/Smt4, or a fragment thereof (for example from Invitrogen, catalog no. 12588-018). In a particular example, sumoase recognizes the tertiary structure of SUMO rather than an amino acid sequence.

Subject: Living multi-cellular vertebrate organisms, a category that includes human and non-human mammals (such as laboratory or veterinary subjects). In one example, a subject is one in need of treatment (such as prevention) of an infectious disease.

Therapeutically effective amount: An amount of an agent that alone, or together with a pharmaceutically acceptable carrier or one or more additional therapeutic agents, induces the desired response. A therapeutic agent, such as one or more AMPs, is administered in therapeutically effective amounts that provide an antimicrobial response (such as at least partial relief from one or more symptoms associated with an infectious disease, for example a fever), for example against a target microbe.

Effective amounts a therapeutic agent can be determined in many different ways, such as assaying for a decrease in one or more symptoms associated with an infection, for example by assaying for improvement of a physiological condition of a subject having an infectious disease. Effective amounts also can be determined through various in vitro, in vivo or in situ assays.

Therapeutic agents can be administered in a single dose, or in several doses, for example weekly, every 2 weeks, monthly, or bimonthly, during a course of treatment. However, the effective amount of can be dependent on the source applied, the subject being treated, the severity and type of the condition being treated, and the manner of administration.

In one example, it is an amount sufficient to partially or completely alleviate symptoms of an infection in a subject. Treatment can involve only slowing the progression of the infection temporarily, but can also include halting or reversing the infection permanently. For example, a pharmaceutical preparation can decrease one or more symptoms of the infection, for example decrease in one or more symptoms by at least 20%, at least 50%, at least 70%, at least 90%, at least 98%, or even at least 100%, as compared to an amount in the absence of the pharmaceutical preparation.

Transduced and Transformed: A virus or vector “transduces” or “transfects” a cell when it transfers nucleic acid molecules into the cell. A cell is “transformed” by a nucleic acid molecule transduced into the cell when the DNA becomes stably replicated by the cell, either by incorporation of the nucleic acid molecule into the cellular genome, or by episomal replication. As used herein, the term transformation encompasses all techniques by which a nucleic acid molecule can be introduced into such a cell, including transfection with viral vectors, transformation with plasmid vectors, and introduction of naked DNA by electroporation, lipofection, and particle gun acceleration.

Transgene: An exogenous nucleic acid sequence supplied by a vector. In one example, a transgene encodes a fusion peptide, such as a SUMO-AMP peptide.

Treating a disease: “Treatment” refers to a therapeutic intervention that ameliorates a sign or symptom of a disease or pathological condition, such a sign or symptom of an infectious disease. Treatment can also induce remission or cure of a condition, such as an infectious disease. In particular examples, treatment includes preventing a disease, for example by inhibiting the full development of a disease, such as preventing development of an infectious disease. Prevention of a disease does not require a total absence of an infection. For example, a decrease in the symptoms associated with the infection of at least 10% or at least 25% can be sufficient.

Under conditions sufficient for: A phrase that is used to describe any environment that permits the desired activity.

In one example, includes cell culture conditions (such as the media and incubation temperature) that permit expression of an exogenous fusion protein in the cell, such as expression of a fusion protein encoded by a vector. In particular examples, includes cell culture conditions that permit expression of a SUMO-AMP fusion protein in the cell, for example without killing the cell.

Vector: A nucleic acid molecule as introduced into a cell, thereby producing a transformed cell. A vector can include nucleic acid sequences that permit it to replicate in the cell, such as an origin of replication. A vector can also include one or more selectable marker genes and other genetic elements known in the art. In particular examples, a vector includes a promoter operably linked to a nucleic acid sequence that encodes for a SUMO-AMP fusion protein.

A vector can transduce, transform or infect a cell, thereby causing the cell to express nucleic acid molecules and/or proteins other than those native to the cell. A vector optionally includes materials to aid in achieving entry of the nucleic acid into the cell, such as a viral particle, liposome, protein coating or the like. In one example, a vector is a viral vector, such as retroviral and adenoviral vectors.

### Methods of Producing Proteins

Antimicrobial peptides (AMPs) are part of the innate immunity defense line in several diverse organisms, including vertebrates, invertebrates, bacteria, and plants. Due to the nature of the peptide, methods of expressing AMPs recombinantly have failed, for example because the inherent antimicrobial activity kills the cells in which AMPs are expressed. The inventors have discovered that expression of AMP fusion proteins, for example by operably linking a SUMO molecule to the AMP, permits recombinant expression of AMP production without significant adverse effects on the host cells. It appears that SUMO “masks” the inherent antimicrobial activity of the AMP, and in some examples can increase the stability of AMP. The SUMO molecule can be cleaved from the fusion protein, thereby releasing biologically active AMP. One skilled in the art will appreciate that other ubiquitin-like proteins be used in place of SUMO, such as Rub1, Hub1, ISG15, Apg12, Apg8, Urm1, Ana 1a, and Ana 1b, wherein the appropriate protease is selected to cleave the fusion protein and release biologically active AMP. Therefore, reference to a SUMO-AMP fusion protein herein includes examples where another ubiquitin like protein is used in place of SUMO.

Based on these observations, methods of producing proteins, such as a SUMO-AMP fusion protein, in a host cell are provided. In one example, the method includes expressing a nucleic acid molecule encoding a fusion protein in a cell, wherein the nucleic acid molecule includes a nucleic acid molecule encoding SUMO operably linked to a nucleic acid molecule encoding an AMP. For example, the nucleic acid molecule encoding SUMO can operably linked upstream to the nucleic acid molecule encoding the antimicrobial peptide (such that SUMO is N-terminal to AMP), or the nucleic acid molecule encoding SUMO can operably linked downstream to the nucleic acid molecule encoding the antimicrobial peptide (such that SUMO is C-terminal to AMP). In particular examples, the nucleic acid molecule also encodes one or more purification tags, such as 6×-His or a FLAG tag. The nucleic acid molecule encoding the one or more purification tags can be operably linked upstream or downstream to the nucleic acid molecule encoding the AMP or SUMO. Ideally, the nucleic acid molecules encoding SUMO and AMP (and in some example also purification tag) are in frame, thus resulting in a properly encoded fusion peptide. The cell is then cultured under conditions that permit expression of the fusion protein. In particular examples, presence of SUMO in the SUMO-AMP fusion protein significantly decreases the antimicrobial activity of the AMP, increases stability of the AMP, or combinations thereof.

Although the term “SUMO-AMP” fusion protein is used throughout this application, this notation does not exclude the possibility of other elements (such as the presence of a purification tag N- or C-terminal to AMP or N- or C-terminal to SUMO) and does not signify that SUMO is N-terminal to AMP (that is, SUMO can be N- or C-terminal to AMP). In addition, SUMO can be replaced with another ubiquitin-like protein. Methods of determining an appropriate nucleic acid molecule to use to encode the desired fusion protein are known in the art.

### SUMO

SUMO (small ubiquitin related modifier) is a small ubiquitin-like protein that becomes covalently conjugated to a variety of target proteins, the large majority of which are found in the nucleus. SUMO is also referred to in the art as Sentrin, SMT3, PIC1, GMP1, and UBL1. In particular examples, the presence of SUMO in a SUMO-AMP fusion protein significantly decreases the native antimicrobial activity of an AMP, increases the stability of the AMP, or both. This activity of SUMO can be referred to as “SUMO activity.” Methods of testing such activities are described herein.

SUMO from any species can be used in the methods provided herein. An exemplary SUMO sequence is shown in SEQ ID NO: 15 (coding sequence shown in SEQ ID NO: 14). However, one skilled in the art will appreciate that SUMO variants that retain SUMO activity can be used. For example, SUMO can have at least 50% sequence identity, for example at least 60%, at least 75%, at least 80%, at least 90%, at least 95%, at least 97%, or at least 98% sequence identity to any publicly available SUMO sequence (such as SEQ ID NOS: 14 and 15).

Variant SUMO protein sequences can be produced by manipulating the nucleotide sequence encoding a SUMO using standard procedures such as site-directed mutagenesis or PCR. In particular examples, variants of SUMO (such as SEQ ID NO: 15) have at least 90% of the native SUMO activity, such as at least 95%, at least 98%, at least 100%, or even at least 150% of the native SUMO activity.

Variants include substitution of one or more amino acids, such as one or more conservative amino acid substitutions, one or more non-conservative amino acid substitutions, or combinations thereof. Variants also include deletion or insertion of one or more amino acids (or combinations thereof, such as a single deletion together with multiple insertions), such as addition or deletion of no more than 20 amino acids, no more than 10 amino acids, no more than 5 amino acids, or no more than 2 amino acids, such as an addition or deletion of 1-2 amino acids, 1-4 amino acids, or 2-20 amino acids in a SUMO sequence. Variants also include fragments of SUMO. In particular examples a fragment of SUMO includes at least 15 contiguous amino acids of a known SUMO sequence, as long as the fragment retains SUMO activity. In one example, a fragment includes at least 30 contiguous amino acids of any of SUMO sequence (such as SEQ ID NO: 15), such as at least 40, at least 50, at least 75, at least 90, or at least 95 contiguous amino acid residues of a SUMO sequence (such as SEQ ID NO: 15), as long as such fragments retain SUMO activity.

In a particular example, a variant AMP sequence includes at least 80% sequence identity to a known SUMO sequence (such as SEQ ID NO: 15), for example at least 85%, at least 90%, at least 95%, at least 97%, at least 98% or at least 99% sequence identity to a known SUMO sequence (such as SEQ ID NOS: 15) as long as the peptide encoded by the amino acid sequence retains SUMO activity.

Exemplary non-conservative and conservative substitutions are discussed below. In one example, a known AMP sequence (such as SEQ ID NO: 15) includes at least one conservative amino acid substitution, such as at least 2, at least 3, at least 4, at least 5, at least 6, at least 8, at least 9, at least 10, at least 11, at least 12, at least 13, at least 14, at least 15, at least 16, at least 17, at least 18, at least 19, or at least 20 conservative amino acid substitutions, for example 1-2, 1-5, 1-10, or 1-20 conservative amino acid substitutions.

The effects of non-conservative or conservative amino acid substitutions (or other deletions or insertions) in a SUMO sequence can be assessed for by determining the ability of the variant SUMO in a SUMO-AMP fusion protein to decrease the antimicrobial activity of the AMP or increase the stability of the AMP in a SUMO-AMP fusion protein. Ideally, variations made to a SUMO sequence do not substantially decrease the desired SUMO activity, and in some examples increase the desired activity of SUMO.

### Antimicrobial Peptides

Antimicrobial peptides (AMPs) are involved in the innate immunity of numerous organisms, including bacteria, fungi, plants, invertebrates, and vertebrates. AMPs exhibit broad-spectrum activity against Gram-positive and Gram-negative bacteria, yeasts, fungi, parasites, and enveloped viruses. AMPs are generally short, such as about 12-100 amino acids (for example 12-50 amino acids), are positively charged (such as net charge of about +2 to +9), and are amphiphilic.

To date, hundreds of AMPs are recognized by those skilled in the art. Particular examples of AMPs include, but are not limited to, cathelicidins, defensins, human epididymis (HE2) peptides, histatins, magainins, mellitins, temporins, dermaseptins, cecropins, as well as numerous others known in the art (for example see Jenssen et al., Clin. Microl. Rev. 19:491-511, 2006 for a review article). In a specific example, the AMP is a defensin, cathelicidin (such as CRAMP), or human epididymis 2 peptide.

In one example, the AMP sequence is a native AMP sequence, such as those shown in SEQ ID NOS: 1-12, 37 and 39. However, one skilled in the art will appreciate that variant AMP sequences can be used, such as variants of SEQ ID NOS: 1-12, 37 and 39 (or fragments thereof such as amino acids 72-103 of SEQ ID NO: 11), which retain antimicrobial activity or even increased antimicrobial activity. For example, substitutions in LL37 (e.g., SEQ ID NO: 1, 37 and 39) can increase the biological activity of LL37, such as replacement of E¹⁶ and K²⁵ with Leu (L), replacement of A²², D²⁶, and N³⁰ with Lys (K), or combinations thereof In another example, substitutions in CRAMP-18 can increase the biological activity of CRAMP (e.g., SEQ ID NO: 2), such as replacement of residues 2, 9, 13, 16 (or combinations thereof) with Lys (K). Exemplary variant CRAMPs are shown in SEQ ID NOS: 41 and 43.

Variant peptide sequences can be produced by manipulating the nucleotide sequence encoding an AMP using standard procedures such as site-directed mutagenesis or PCR. In particular examples, variants of an AMP (such as a variant of the sequence shown in any of SEQ ID NOS: 1-12, 37, 39, 41 and 43, or fragments thereof such as amino acids 72-103 of SEQ ID NO: 11) have at least 90% of the native antimicrobial activity, such as at least 95%, at least 98%, at least 100%, or even at least 150% of the native antimicrobial activity. In a particular example, a native AMP sequence is modified to include at least one amino acid substitution that increases the antimicrobial activity of the peptide.

Variants include substitution of one or more amino acids, such as one or more conservative amino acid substitutions, one or more non-conservative amino acid substitutions, or combinations thereof. Variants also include deletion or insertion of one or more amino acids (or combinations thereof, such as a single deletion together with multiple insertions), such as addition or deletion of no more than 15 amino acids, no more than 10 amino acids, no more than 5 amino acids, or no more than 2 amino acids, such as an addition or deletion of 1-2 amino acids, 1-4 amino acids, or 2-10 amino acids in an AMP sequence. Variants also include fragments of AMPs. In particular examples a fragment of AMP includes at least 15 contiguous amino acids of a known AMP sequence, as long as the fragment retains antimicrobial activity. In one example, a fragment includes at least 15 contiguous amino acids of any of AMP sequence (such as an AMP sequence shown in any of SEQ ID NOS: 1-12, 37, 39, 41, 43), such as at least 16, at least 17, at least 18, at least 19, at least 20, at least 25, at least 30, at least 40, or at least 50 contiguous amino acid residues of an AMP sequence (such as an AMP sequence shown in any of SEQ ID NOS: 1-12, 37, 39, 41, and 43), as long as such fragments retain antimicrobial activity. On exemplary fragment is amino acids 72-103 of SEQ ID NO: 11.

In a particular example, a variant AMP sequence includes at least 80% sequence identity to a known AMP sequence (such as any of SEQ ID NOS: 1-12, 37, 39, 41, and 43, or a fragment thereof such as amino acids 72-103 of SEQ ID NO: 11), for example at least 85%, at least 90%, at least 95%, at least 97%, at least 98% or at least 99% sequence identity to a known AMP sequence (such as any of SEQ ID NOS: 1-12, 37, 39, 41, and 43 or a fragment thereof such as amino acids 72-103 of SEQ ID NO: 11) as long as the peptide encoded by the amino acid sequence retains antimicrobial activity.

Non-conservative substitutions are those wherein the amino acids have more substantial difference, such as their effect on maintaining: (a) the structure of the polypeptide backbone in the area of the substitution, for example, as a sheet or helical conformation; (b) the charge or hydrophobicity of the polypeptide at the target site; or (c) the bulk of the side chain. The substitutions that in general are expected to produce the greatest changes in peptide function are those in which: (a) a hydrophilic residue, such as serine or threonine, is substituted for (or by) a hydrophobic residue, such as leucine, isoleucine, phenylalanine, valine or alanine; (b) a cysteine or proline is substituted for (or by) any other residue; (c) a residue having an electropositive side chain, such as lysine, arginine, or histidine, is substituted for (or by) an electronegative residue, such as glutamic acid or aspartic acid; or (d) a residue having a bulky side chain, such as phenylalanine, is substituted for (or by) one not having a side chain, such as glycine.

In contrast, conservative substitutions are those wherein the amino acids have similar biochemical properties. In one example, an AMP sequence (such as SEQ ID NOS: 1-12, 37, 39, 41 or 43, or a fragment thereof such as amino acids 72-103 of SEQ ID NO: 11) includes at least one conservative amino acid substitution, such as at least 2, at least 3, at least 4, at least 5, at least 6, at least 8, at least 9, at least 10, at least 11, at least 12, at least 13, at least 14, at least 15, at least 16, at least 17, at least 18, at least 19, or at least 20 conservative amino acid substitutions, for example 1-2, 1-5, 1-10, or 1-20 conservative amino acid substitutions.

The effects of conservative or non-conservative amino acid substitutions (or other deletions or insertions) in an AMP sequence can be assessed for antimicrobial activity using assays known in the art. Ideally, changes to an AMP sequence do not substantially decrease the antimicrobial activity of the AMP, and in some examples increase the antimicrobial activity of the AMP.

### Purification Tags

The disclosed fusion proteins can further include one or more purification tags. Such tags can be used to permit purification of the SUMO-AMP fusion protein, or the cleaved AMP molecule, for example using affinity chromatography. The purification tag can be located at the N- or C-terminus of SUMO-AMP, or between SUMO and AMP in the SUMO-AMP fusion (such as SUMO-Tag-AMP). Methods of generating such recombinant proteins are known in the art of molecular biology.

For example, purification tags can provide an epitope, allowing an antibody to recognize the fusion protein. The antibody therefore can be coupled to a solid medium (such as sepharose or agarose), thereby permitting purification of the antigen (such as GST or 6×-His) from the cellular lystate. Briefly, the cellular lysate is incubated under conditions sufficient for SUMO-AMP or AMP containing a purification tag to bind to the antibody attached to the solid medium. Non-bound proteins are washed away, and the bound proteins eluted. The eluate is collected. If desired, the purification tag can be removed.

In another example, the purification tag permits it to be selected for affinity binding to a particular affinity agent. The agent therefore can be coupled to a solid medium (such as sepharose or agarose), thereby permitting purification of the SUMO-AMP or AMP from the cellular lystate via the particular protein tag. Briefly, the cellular lysate is incubated under conditions sufficient for SUMO-AMP or AMP containing a purification tag to bind to the affinity agent attached to the solid medium. Non-bound proteins are washed away, and the bound proteins eluted. The eluate is collected. If desired, the protein tag can be removed. For example, His-tags have an affinity for nickel or cobalt ions which can be covalently bound to NTA (nitrilotriacetic acid) for the purposes of solid medium entrapment. For elution, a nickel chelating agent, such as imidazole, can be used. GST has an affinity for glutathione (such as glutathione sepharose). For elution, excess glutathione is used to displace the tagged protein.

Particular non-limiting examples of purification tags include biotin carboxyl carrier protein (BCCP) tag, c-myc-tag, calmodulin-tag, FLAG-tag, His-tag (such as 6×-His), maltose binding protein-tag, Nus-tag, Glutathione-S-transferase (GST)-tag, green fluorescent protein-tag, thioredoxin-tag, S-tag, and Strep-tag. One skilled in the art will appreciate that combinations of these tags can be used, such as 2, 3, or 4 of these tags.

### Removal of SUMO

The resulting SUMO-AMP fusion protein generated using the methods provided herein can be incubated with appropriate agents that permit removal of SUMO (or other ubiquitin-like protein that is used in place of SUMO) from the AMP. In some examples, cleavage of the fusion protein and removal of SUMO restores antimicrobial activity to the liberated AMP. Such restoration does not need to be 100%. For example, restoration of at least 20%, at least 50%, at least 70%, or at least 90% of the native antimicrobial activity can be sufficient. Methods of measuring AMP activity are known in the art (for example, see Example 3).

Methods of removing SUMO from a SUMO fusion protein are known in the art. For example, the SUMO-AMP fusion protein can be incubated with a protease, such as sumoase, under conditions that permit the enzyme to cleave SUMO from the fusion protein, thereby liberating the AMP. Sumoase is commercially available, and can be used as directed by the manufacturer.

In some examples, SUMO is removed from the fusion protein in vitro. For example, the method can include isolating the SUMO-AMP fusion protein from the cell, and then incubating the fusion protein in the presence of a protease. In some examples, SUMO is removed from the fusion protein in vivo. For example, the method can include expressing an appropriate enzyme in the cell, such as a native or exogenous sumoase, thereby permitting cleavage in vivo. The resulting AMP can then be purified from the cell.

### Protein Isolation

Methods of isolating proteins from cells are well known in the art. In some examples, the SUMO-AMP fusion protein is isolated from the cell. In other examples, the SUMO-AMP fusion protein is cleaved in vivo to release AMP, and the AMP can be purified from the cell.

In some examples, the cell is lysed, and the proteins substantially separated from other cellular material (such as nucleic acid molecules). The resulting protein mixture, which includes SUMO-AMP or AMP, is subjected to conditions that permit isolation of the desired SUMO-AMP or AMP. For example, the cellular protein mixture can be subjected to SDS-PAGE, and the band containing AMP isolated. Exposure of the cleaved fusion protein to SDS-PAGE can also be used to confirm cleavage of the fusion protein.

In another example, affinity chromatography is used to purify or isolate the SUMO-AMP or AMP from the cellular lysate. Affinity chromatography permits purification of the desired protein, based on a specific biologic interaction, such as that between antigen and antibody, enzyme and substrate, or receptor and ligand. For example, a cell lysate can be incubated in the presence of a solid medium, wherein the solid medium (such as beads, agarose, or sepharose) includes an affinity agent that specifically binds to SUMO-AMP or AMP. Ideally, the affinity agent binds to SUMO-AMP or AMP with significantly higher affinity than to other molecules. After allowing the SUMO-AMP or AMP to bind to the solid medium via the affinity agent, unbound proteins can be washed away, the SUMO-AMP or AMP eluted from the solid medium. Purity of the resulting peptide can be confirmed using methods known in the art, such as SDS-PAGE.

In one example the affinity agent is an antibody (such as a monoclonal or polyclonal antibody) that is highly specific for SUMO or AMP. In some examples, the SUMO-AMP fusion protein includes an affinity or purification tag, such as a GST tag attached to the N- or C-terminus of the fusion protein (or between SUMO and AMP in the fusion protein), and the antibody can be one that specifically binds to the purification tag. In other examples, the affinity agent can be an appropriate complement to the purification tag. For example, a fusion protein containing a FLAG tag can be purified by incubation with Ni-agarose.

### Host Cells

Recombinant cells that express a SUMO-AMP fusion protein can be eukaryotic or prokaryotic. Host cells express the exogenous SUMO-AMP fusion protein, for example via a vector encoding the protein. In some examples, the host cells express an endogenous sumoase or an exogenous sumoase (such as one expressed by a recombinant nucleic acid molecule), for example to permit cleavage of the SUMO-AMP protein in vivo. Ideally, such host cells permit efficient expression of the SUMO-AMP fusion protein.

Examples of cells that can be used as host cells include, but are not limited to bacterial cells (such as Lactobacillus, Lactococcus, Bacillus, Escherichia, Geobacillus, Corynebacterium, or Clostridium), fungal cells (such as Aspergillus or Rhizopus cells), plant cells (such as corn, wheat, rice, or soybean cells), yeast cells, and mammalian cells. In one example, a cell is a microorganism. The term “microorganism” refers to any microscopic organism including, but not limited to, bacteria, algae, fungi, and protozoa. Thus, E. coli, B. subtilis, S. cerevisiae, Kiuveromyces lactis, Candida blankii, Candida rugosa, and Pichia pastoris are microorganisms that can be used.

### Antimicrobial Fusion Proteins and Nucleic Acid Molecules

The present disclosure provides novel isolated fusion peptides and nucleic acid sequences that encode such proteins. In one example, a fusion protein includes a SUMO amino acid sequence linked (directly or indirectly) to an AMP amino acid sequence. In some examples, the fusion protein further includes one or more purification tags. In particular examples, such fusion peptides are produced using the methods disclosed herein.

### Proteins

Fusion peptides that include a SUMO sequence and an AMP sequence are disclosed herein. The disclosed SUMO-AMP fusion peptides can be used to produce AMP, for example to produce therapeutic amounts of AMP, which in some examples are used to treat a subject having an infection.

In particular examples, the antimicrobial activity of the fusion protein is significantly reduced relative to the native antimicrobial activity of the AMP. In particular examples, the antimicrobial activity of the fusion protein is reduced by at least 20%, at least 50%, at least 80%, at least 90% or even at least 95%, relative to the native AMP. In particular examples, antimicrobial activity is determined by measuring the viability of bacteria (or other target cell) in the presence of the SUMO-AMP fusion protein or the native AMP (for example by measuring the optical density of the bacteria over time, see Example 3).

In another example, the stability of the SUMO-AMP fusion protein is significantly increased relative to the native stability of the AMP. In particular examples, enhancing the stability of a protein protects the protein from misfolding or unfolding. In particular examples, the stability of the fusion protein is increased by at least 20%, at least 50%, at least 80%, at least 90% or even at least 95%, relative to the native AMP. For example, the stability of the fusion protein can be evidenced by a decrease in misfolding or unfolding of the AMP by at least 20%, at least 50%, at least 80%, at least 90% or even at least 95%, relative to the native AMP. Measuring protection from unfolding can be measured using methods known in the art. In one example, for example circular dichroism can be used to measure the α-helix content of the peptide (wherein the active peptide is an amphipathic α-helix), for example using the methods disclosed in Matsuo et al., J. Biochem.135:405-11, 2004 (herein incorporated by reference as to the methods). In particular examples, the structural transitions of a protein (such as AMP or SUMO-AMP) as it is unfolded by heat or chemical denaturants such as urea, are monitored spectrophotometrically. The more stable a protein is, the more urea or higher temperature is needed to unfold the protein. In one example, the fluorescence emission of tryptophan (W) at 295 nm can be monitored if a Trp amino acid is present in the protein. Otherwise, other spectra can be measured.

Fusion proteins (and their corresponding nucleic acid coding sequence) can be represented by the sequence X—Y or Y—X, wherein X and Y represent different protein sequences (see the Examples listed in Table 1). Methods of generating the fusion proteins described in Table 1, for example using recombinant technologies, are well known in the art.

TABLE 1
Exemplary SUMO-AMP fusion proteins

| | Representative | Exemplary |
|---|---|---|
| Example | sequence* | SEQ ID NO: |
| 1 | X-Y | 22 |
| 2 | Y-X | 23 |
| 3 | X-L-Y | 24 |
| 4 | Y-L-X | |
| 5 | X-Y-P | 25 |
| 6 | Y-X-P | |
| 7 | X-P-Y | 26 |
| 8 | P-X-Y | 27, 40, 47, 49, 51 |
| 9 | P-Y-X | 28 |
| 10 | Y-P-X | |
| 11 | X-L-Y-P | 29 |
| 12 | Y-L-X-P | |
| 13 | P-L-X-Y | 30 |
| 14 | P-L-Y-X | 31 |
| 15 | Y-P-L-X | |
| 16 | P-X-L-Y | 32 |
| 17 | P-X-Y-P | 45 |

*X is SUMO or other ubiquitin like protein (such as SEQ ID NO: 15 or a variant thereof); Y is an AMP (such as any of SEQ ID NOS: 1-12, 37, 39, 41, 43 or variants thereof); L is a linker of 1-50 amino acids; P is a purification tag (such as any of those described herein).

For example, if X is SUMO and Y is an AMP, the fusion peptide can be represented as X—Y (where SUMO is linked to the N-terminus of AMP) or Y—X (where SUMO is linked to the C-terminus of AMP). In such examples, SUMO is directly linked to the AMP. For example, SUMO can be directly linked to the N-terminus of an AMP (Example 1 in Table 1). However, the term “SUMO-AMP” as used herein does not specify the location of SUMO relative to AMP, and does not indicate that other elements (such as a purification tag) are not present. For example, SUMO can be directly linked to the C-terminus of an AMP (Example 2 in Table 1).

In other examples, fusion proteins include linkers (L) represented by the sequence X-L-Y or Y-L-X (see Examples 3 and 4 and 11-16 in Table 1). In such examples, SUMO (X) is indirectly linked to the AMP (Y), for example via a spacer or linker peptide (L). Particular examples of spacers include on or more alanine or 5 glycine residues, or other nonpolar amino acids or neutral polar amino acids. In some examples, spacers are no more than 50 amino acids, such as no more than 20 amino acids, no more than 10 amino acids, or no more than 5 amino acids, for example 5-10, 5-15, or 5-50 amino acids.

The disclosed fusion proteins can also include purification tags (P), for 10 example represented by the sequence X—P—Y, Y—P—X, P—X—Y—P, and P—X—Y (see Examples 5-17 in Table 1). In such examples, the purification tag can be attached to SUMO (X), AMP (Y), or both.

In some examples, the disclosed fusion proteins include purification tags (P) and linkers (L), for example represented by the sequence P—X-L-Y or P—Y-L-X (see Examples 11-16 in Table 1. Although only a few examples are shown, one skilled in the art will appreciate that additional combinations can be made.

Particular examples of SUMO-AMP fusion peptides are shown in SEQ ID NOS: 22-32, 40, 45, 47, 49, and 51. However, one skilled in the art will appreciate that other SUMO-AMP fusion proteins can be generated using known SUMO sequences (such as SEQ ID NO: 15) and known AMP sequences (such as SEQ ID NOS: 1-12, 37, 39, 41, 43, or a fragment thereof such as amino acids 72-103 of SEQ ID NO: 11), as well as known purification tag and linker sequences. In addition, the disclosure encompasses SUMO-AMP fusion proteins that include variants of known SUMO and AMP sequences (discussed in detail above). For example, a SUMO-AMP can have at least 80% sequence identity, for example at least 85%, at least 90%, at least 95%, at least 97%, or at least 98% sequence identity to a SUMO-AMP sequence (such as those shown in SEQ ID NOS: 22-32, 40, 45, 47, 49, and 51).

Variant SUMO-AMP protein sequences can be produced by manipulating the nucleotide sequence encoding a SUMO-AMP using standard procedures such as site-directed mutagenesis or PCR. Variants include substitution of one or more amino acids, such as one or more conservative amino acid substitutions, one or more non-conservative amino acid substitutions, or combinations thereof. Variants also include deletion or insertion of one or more amino acids (or combinations thereof, such as a single deletion together with multiple insertions), such as addition or deletion of no more than 20 amino acids, no more than 10 amino acids, no more than amino acids, or no more than 2 amino acids, such as an addition or deletion of 1-2 amino acids, 1-4 amino acids, or 2-20 amino acids in a SUMO-AMP sequence. Variants also include fragments of SUMO-AMP. In particular examples a fragment of SUMO includes at least 50 contiguous amino acids of a SUMO-AMP sequence, as long as the fragment retains SUMO-AMP activity. In one example, a fragment includes at least 50 contiguous amino acids of any SUMO-AMP sequence (such as any of SEQ ID NOS: 22-32, 40, 45, 47, 49, and 51), such as at least 75, at least 90, or at least 95 contiguous amino acid residues of a SUMO-AMP sequence (such as any of SEQ ID NOS: 22-32, 40, 45, 47, 49, and 51), as long as such fragments retain SUMO-AMP activity.

Exemplary non-conservative and conservative substitutions are discussed above. In one example, a SUMO-AMP sequence (such as any of SEQ ID NOS: 22-32, 40, 45, 47, 49, and 51) includes at least one conservative amino acid substitution, such as at least 2, at least 3, at least 4, at least 5, at least 6, at least 8, at least 9, at least 10, at least 11, at least 12, at least 13, at least 14, at least 15, at least 16, at least 17, at least 18, at least 19, or at least 20 conservative amino acid substitutions, for example 1-2, 1-5, 1-10, or 1-20 conservative amino acid substitutions.

The effects of non-conservative or conservative amino acid substitutions (or other deletions or insertions) in a SUMO-AMP sequence can be assessed for by determining the ability of the variant SUMO-AMP to decrease the antimicrobial activity of the AMP or increase the stability of the AMP in a SUMO-AMP fusion protein. Ideally, variations made to a SUMO-AMP sequence do not substantially decrease the desired SUMO-AMP activity, and in some examples can even increase the desired activity of SUMO-AMP. In particular examples, variants of SUMO-AMP (such as any of SEQ ID NOS: 22-32, 40, 45, 47, 49, and 51) have at least 90% of the native SUMO-AMP activity (such as decreased antimicrobial activity, increased stability, or both), such as at least 95%, at least 98%, at least 100%, or even at least 150% of the native SUMO-AMP activity.

### Nucleic Acid Molecules

Also disclosed are isolated nucleic acid molecules that encode SUMO-AMP fusion peptides, for example a sequence which encodes any of SEQ ID NOS: 22-32, 40, 45, 47, 49, and 51. However, the disclosure also encompasses nucleic acid molecules that encode variants, fusions, and fragments of any of SEQ ID NOS: 22-32, 40, 45, 47, 49, and 51, which retain the ability to encode a protein having SUMO-AMP activity, or even increased SUMO-AMP activity.

In one example an isolated nucleic acid molecule encoding a SUMO-AMP is operably linked to a promoter sequence, and can be part of a vector, such as a bacterial expression vector. In particular examples, the vector further includes a promoter to direct expression of the fusion peptide, as well as a selectable marker sequence (such as a sequence coding for antibiotic resistance). The nucleic acid can be a recombinant nucleic acid that can be used to transform cells and make transformed cells.

The disclosed nucleic acid molecules and vectors can be used to transform a host cell (such as those described above), thereby permitting expression of one or more SUMO-AMP fusion proteins in the cell. Therefore, the present disclosure provides transformed cells that include a nucleic acid molecule that encodes for SUMO-AMP (such as encodes for any of SEQ ID NOS: 22-32, 40, 45, 47, 49, and 51 or variants thereof). In one example, such a transformed cell produces SUMO-AMP, wherein the fusion protein has significantly decreased antimicrobial activity relative to the native AMP. Transformed cells can be eukaryotic or prokaryotic, such as bacterial cells, fungal cells, or yeast cells. Specific examples of transgenic cells include Lactobacillus, Lactococcus, Bacillus, or Escherichia cells.

Nucleic acid sequences that encode variant SUMO-AMP fusion proteins are disclosed herein. In particular examples, variant nucleic acid sequences do not alter the reading frame. For examples, variants can contain a single substitution, multiple insertions (such as multiples of threes), multiple deletions (such as multiples of threes), multiple substitutions, or any combination thereof (such as a single substitution together with multiple insertions) as long as the peptide encoded thereby is a SUMO-AMP. In particular examples, the disclosed isolated nucleic acid molecules share at least 60%, at least 70%, at least 75%, at least 80%, at least 85%, at least 90%, at least 95%, at least 97%, at least 98%, or at least 99% sequence identity with a sequence encoding a SUMO-AMP (such as any of SEQ ID NOS: 22-32, 40, 45, 47, 49, and 51), as long as the peptide encoded by the nucleic acid retains SUMO-AMP activity.

The coding region of a SUMO-AMP sequence can be altered by taking advantage of the degeneracy of the genetic code to alter the coding sequence in such a way that, while the nucleic acid sequence is substantially altered, it nevertheless encodes a peptide having an amino acid sequence identical or substantially similar to the native amino acid sequence. For example, codon preferences and codon usage tables for a particular species can be used to engineer isolated nucleic acid molecules that take advantage of the codon usage preferences of that particular species (such as the codon usage preference of a host cell). Because of the degeneracy of the genetic code, alanine is encoded by the four nucleotide codon triplets: GCT, GCA, GCC, and GCG. Thus, the nucleic acid sequence of the open reading frame can be changed at an alanine position to any of these codons without affecting the amino acid sequence of the encoded SUMO-AMP or the characteristics of the SUMO-AMP. Based upon the degeneracy of the genetic code, nucleic acid variants can be derived from a nucleic acid sequence using standard DNA mutagenesis techniques or by synthesis of nucleic acid sequences. Thus, this disclosure also encompasses nucleic acid molecules that encode the same SUMO-AMP but vary in nucleic acid sequence by virtue of the degeneracy of the genetic code. Therefore, the SUMO-AMP nucleic acid sequences disclosed herein can be designed to have codons that are preferentially used by a particular cell of interest.

In a particular example, a nucleic acid molecule encoding a SUMO-AMP fusion protein is at least 300 nucleotides (nt) in length, such as at least 400 nt, at least 500 nt, at least 600 nt, or at least 1000 nt (such as 600-4500 nt), and can hybridize under highly stringent hybridization conditions to a nucleic acid molecule encoding any of SEQ ID NOS: 22-32, 40, 45, 47, 49, and 51. Exemplary highly stringent hybridization conditions include when the hybridization is performed at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 65° C. with a wash solution containing 0.2×SSC and 0.1% sodium dodecyl sulfate.

SUMO-AMP fusion peptides and nucleic acid molecules encoding such peptides can be produced by standard recombinant molecular biology methods. Details of these techniques are provided in Sambrook et al. (ed.), Molecular Cloning: A Laboratory Manual 2nd ed., vol. 1-3, Cold Spring Harbor Laboratory Press, Cold Spring, Harbor, N.Y., 1989, Ch. 15. Nucleic acid molecules can contain changes of a coding region to fit the codon usage bias of the particular organism (such as a host cell) into which the molecule is to be introduced.

### Kits

Kits are provided that include one or more of the disclosed SUMO-AMP fusion proteins, nucleic acid molecules that encode such proteins (such as a vector including such a nucleic acid molecule), or combinations thereof. The kit can also include a protease capable of cleaving the SUMO from the AMP (such as sumoase), cells suitable for expression of the nucleic acid molecules, or combinations thereof. The kit can further include a lysis buffer, a wash buffer, an elution buffer, or combinations thereof.

### Methods of Treatment

The present disclosure also provides methods of treating an infection in a subject using AMP produced using the methods provided herein. For example, the method can include administering a therapeutically effective amount of one or more AMPs produced by the methods provided herein to a subject in need thereof, thereby treating the infection. Exemplary infections include infection with Pseudomonas aeruginosa, Staphylococcus, Citrobacter rodentium, EPEC/EHEC, and Vaccinia virus. In some examples, the subject is pre-screened to determine if they have an infection that can be treated with a particular AMP.

The particular mode of administration can be determined by a skilled clinician. In specific examples, the one or more AMP molecules are administered via injection (such as i.v.) or orally. If desired, multiple doses can be administered over a time period.

In addition, the therapeutic dose can be determined by a skilled clinician. In a specific example, the dosage is about 1-1000 μM per gram weight of the subject, such as about 10-100 μM per gram weight of the subject, for example 50 μM per 30 grams of weight of the subject.

### Example 1

### Generation of SUMO-AMP Fusion Proteins

This example describes methods used to generate a fusion protein that includes SUMO fused to the N-terminus of CRAMP (wild-type or mutated) or the N-terminus of LL37. However, one skilled in the art will appreciate that similar methods can be used to generate similar fusion proteins for any AMP of interest, such as a defensin, by substituting the CRAMP or LL37 sequence with the other AMP sequence. Similarly, these methods can be used to operably link the SUMO to the C-terminus of the AMP.

### SUMO-CRAMP

The full-length CRAMP (mouse specific cathelicidin-related antimicrobial peptide) RNA was isolated from tissue-cultured mouse mast cells using the Qiagen RNA purification protocol. CRAMP cDNA was created using the RT-kit from Ambion. The cDNA was used in a PCR reaction using the primers below to amplify the full-length gene of CRAMP and to insert a FLAG purification tag on the C-terminus of the CRAMP gcgcgggcatatgGGACTTCTCCGCAAAGGTGG (SEQ ID NO: 16, 5′crampsNde);
gcgcgctcgagCTAgcacttgtcgtcgtcgtccttgtagtctttttcttcttcttcCTGCTCCGGCTGAGGT ACAAGT (SEQ ID NO: 17, 3′crampasXholflag);
gcgcgctcgagCTACTGCTCCGGCTGAGGTACAAG (SEQ ID NO: 18, 3′crampasXho1);
GCGCGCCATATGGGACTTCTCCGCgAAGGTGGGGAGgAGATT (SEQ ID NO: 19, pramcsNdel); and
CTACTGCTCCGGCTGAGGTACAAGTTcCTGAAAAAAATTCTcAATTTcCTG GCCAATTTcCTcAAGCTcTTCACC (SEQ ID NO: 20, pramcas)

Using the full-length CRAMP as a template, only the active part of cramp (102 bp), which equals the last 34 aa of the full-length CRAMP protein (SEQ ID NO: 2), was N-terminally fused to SUMO (SEQ ID NO: 14; SUMO protein shown in SEQ ID NO: 15) using the restriction enzymes Nde1 and Xho1, which corresponds to the pet28b vector of Novagen. The vector was modified to harbor SUMO as an N-terminal fusion tag and carries a kanamycin resistance cassette. The construct was transformed into competent BL21 (DElysE) and clones were checked for positive insertion. The resulting HIS tag-SUMO-CRAMP-FLAG-tag protein sequence is shown in SEQ ID NO: 45. The gene was sequenced and positive clones were selected for expression. The same procedure was used for all mutant or variants of CRAMP, including the negative control pramc (SEQ ID NO: 21).

The vector that encoded the SUMO-CRAMP fusion was expressed in E.coli bacteria as follows. A 5 ml culture in LBₖₐₙ was inoculated overnight. The next day, the inoculum was used to start either 100 ml or 500 ml LBₖₐₙ. The cultures were inoculated at 1:500 and were grown at 30 C until it reached OD₆₀₀ of 0.5, then protein expression was induced using 0.4 mM IPTG for 4 hours. Cells were harvested and frozen overnight.

Similar methods were also used to generate a Hi tag-SUMO-CRAMP fusion protein without a FLAG-tag (SEQ ID NO: 47), wherein the primers did not have FLAG-tag encoding sequences.

### SUMO-LL37

The full-length LL37 (human specific cathelicidin) cDNA was isolated from human bone marrow cDNA library provided by Clontech. The cDNA library was used in a PCR reaction using the primers below to amplify a full-length gene of LL37. gcgcaattccatATGAAGACCCAAAGGGATGGCCACTC (SEQ ID NO: 33, hLL37fullNde1); gcgcctcgagCTAGGACTCTGTCCTGGGTACAAGAT (SEQ ID NO: 34, hLL37fullXhoas);
GCGCGCCATATGCTGCTGGGTGATTTCTTCCGGAA (SEQ ID NO: 35, 1137ndeshort).

The resulting active portion of LL37 was cloned (SEQ ID NO: 36, corresponding protein sequence shown in SEQ ID NO: 37). Using the full-length LL37 as a template, only the active part of cramp (114 bp; SEQ ID NO: 38), which equals the last 38 amino acids of a full-length LL37 protein (SEQ ID NO: 39), was N-terminally fused to SUMO (SEQ ID NO: 14) using the restriction enzymes Nde1 and Xho1, which corresponds to the pet28b vector of Novagen. The vector was modified to harbor SUMO as an N-terminal fusion tag and carries a kanamycin resistance cassette. The protein sequence of HisTag-SUMO-LL37 is shown in SEQ ID NO: 40. The construct was transformed into competent BL21 (DElysE) and clones were checked for positive insertion. The gene was sequenced and positive clones were selected for expression.

The vector encoding the SUMO-LL37 fusion was expressed in E. coli as follows. A 5 ml culture in LBₖₐₙ was inoculated overnight. The next day, the inoculum was used to start either 100 ml or 500 ml LBₖₐₙ. The cultures were inoculated at 1:500 and were grown at 30 C until it reached OD₆₀₀ of 0.5, then protein expression was induced using 0.4 mM IPTG for 4 hours. Cells were harvested and frozen overnight.

### SUMO-Mutated CRAMP

CRAMP peptides were mutated using site-directed mutagenesis with the active cramp (SEQ ID NO: 2) as a template. Mutations were made to the CRAMP sequence based on an LL37 sequence. Positions refer to SEQ ID NO: 2, with the first amino acid the wild-type residue and the second amino acid the substituted residue. For mutant CRAMP-M1 the following five amino acid substitutions were made. G1D; L2F; L3F; G6S; and G7K. The resulting mutant CRAMP-M1 amino acid sequence is shown in SEQ ID NO: 41. To generate SEQ ID NO: 41, the following primer was used: GCGCGCCATATGgatttcttccgcaaatctaaagagaagatt (crampm1Nde1; SEQ ID NO 42). For mutant CRAMP-M2 the following four amino acid substitutions were made: K27N; Q31R; P32T; Q34S. The resulting mutant CRAMP-M2 amino acid sequence is shown in SEQ ID NO: 43. To generate SEQ ID NO: 43, the following primer was used:
gcgcctcgagCTAggactctgtccggggtacaagatt (crampM2asXho1; SEQ ID NO: 44).

The mutant CRAMP-M1 and CRAMP-M2 peptides were cloned, fused to SUMO, and expressed as described above for CRAMP. The resulting fusion protein sequences (His tag-SUMO-CRAMP-M1 and His tag-SUMO-CRAMP-M2) are shown in SEQ ID NOS: 49 and 51, respectively.

### Example 2

### Protein Purification

This example describes methods used to purify the SUMO-AMP fusion proteins generated in Example 1 (e.g., SEQ ID NOS: 40, 45, 47, 49, and 51), and digest them with sumoase to release functional AMP (e.g., SEQ ID NOS: 53, 46, 48, 50, and 52, respectively). One skilled in the art will appreciate that similar methods can be used to purify similar fusion proteins for any AMP of interest, such as a defensin.

Protein pellets were dissolved in 1/50 of the original volume of lysis buffer (50 mM NaH₂PO₄ pH 8.0+300 mM NaCl+10 mM Imidazole) and sonicated at position 3 (Fisher ultrasonicator) for either 3×30 sec (trial volume) constant or 8×30 sec constant with ice cooling. The resulting lysis solution was centrifuged at 13000 rpm for 10 minutes, the clear supernatant was considered crude extract containing sumo-cramp and added to either 1 ml (trial run) of Ni—NTA equilibrated in lysis buffer or 2.5 ml of Ni—NTA equilibrated in lysis buffer.

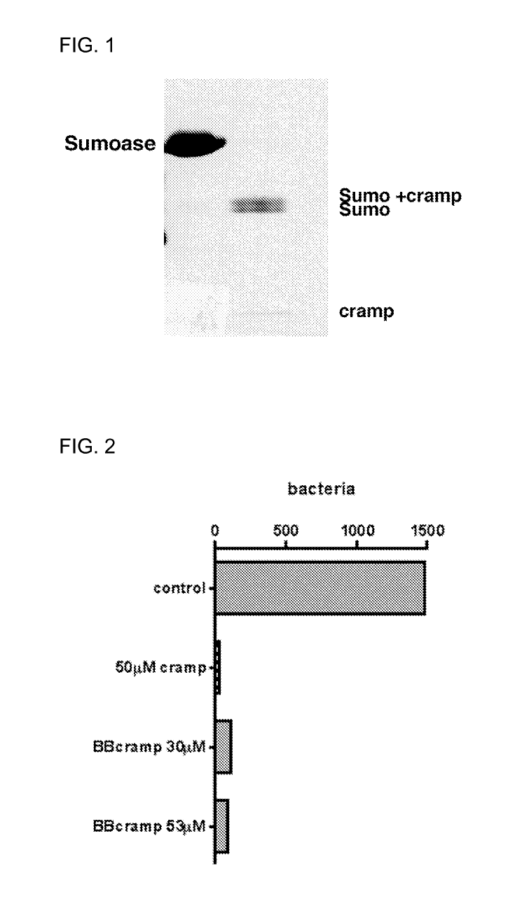

The slurry was rotated at 4° C. for at least 1 hour and then poured into a small gravity column (BioRAD). The column was washed with 20× column volume of washing buffer (50 mM NaH₂PO₄ pH 8.0+300 mM NaCl+20 mM Imidazole) using gravity flow. After washing, the columns were transferred to eppendorf tubes and purified cramp was eluted in 0.5 ml fractions using elution buffer (50 mM NaH₂PO₄ pH 8.0+300 mM NaCl+250 mM Imidazole). Protein purity was assessed using 15% SDS PAGE followed by Coomassie staining (FIG. 1).

The SUMO-CRAMP (native and mutant) and SUMO-LL37 fusion protein concentration was determined using the Lowry assay and subsequently was digested with sumoase to cleave the protective group SUMO from the active CRAMP or LL37 peptide. Briefly, the eluted protein was dialyzed against elute buffer containing no imidazole and then 8 μg sumoase was added for 1 hour at 25° C. The solution was then directly used in functionality assays at different CRAMP or LL37 concentrations (see Example 3).

The resulting AMPs when His tag-SUMO-CRAMP-FLAG tag, His tag-SUMO-CRAMP, and His tag-SUMO-LL37 were digested with sumoase are shown in SEQ ID NOS: 46, 48, and 53, respectively. SEQ ID NO: 53 includes the N-terminal His and Met resulting from proteolytic cleavage after sumoase treatment, thus SEQ ID NO: 53 shows the native LL37 peptide sequence plus the two additional amino acids in the N-terminus. The resulting AMPs when His tag-SUMO-CRAMP-M1 and His tag-SUMO-CRAMP-M2 were digested with sumoase are shown in SEQ ID NOS: 50 and 52, respectively.

In some examples, the protein concentration for the SUMO-AMP peptide from the partial purification after Ni—NTA and sumoase treatment were estimated using a 16% tricine peptide gel (see FIG. 4). The peptide concentration was assumed to be 10% of the total protein content and is equal for all new peptides.

### Example 3

### Functionality Assay

This example describes methods used to demonstrate that the resulting CRAMP and LL37 peptides (e.g., SEQ ID NOS: 46, 48, 50, 52 and 53) generated in Example 2 had antimicrobial activity. One skilled in the art will appreciate that similar methods can be used to confirm the functionality of any AMP of interest, such as a defensin.

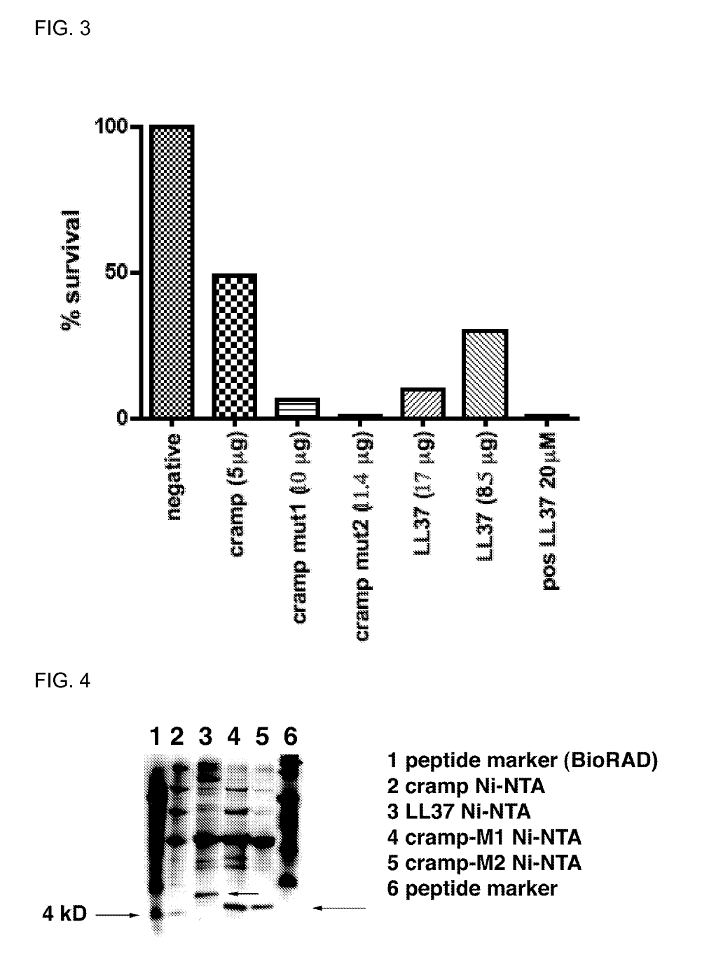

The OD₆₀₀ of a Citrobacter rodentium overnight culture was measured and a dilution series was performed. 100 μl of a 7.1×10² bacteria/ml dilution was used in the functionality assay. For CRAMP, the control culture had an additional lysis buffer+sumoase added, the CRAMP positive control had 50 μM CRAMP (Sigma Genosys) added and CRAMP peptide obtained in Example 2 was added at 30 μM or 53 μM (FIG. 2). For LL37, the positive control had 20 μM pure synthetic LL37 (Microchem Facility, Emory) added, and LL37 peptide obtained in Example 2 was added at 8.5 μg (250 μl) or 17 μg (500 μl) (FIG. 3). 20 μM concentration of the synthetic peptides equals 8.8 μg added.

As shown in FIGS. 2 and 3, the CRAMP and LL37 peptides produced using the methods disclosed herein were almost as efficient in killing Citrobacter rodentium as the commercially available peptide. It is possible that the decrease in efficacy of CRAMP-FLAG-tag is due to the attached FLAG tag, which can be eliminated by removing the FLAG tag using routine methods (for example using a protease such as thrombin or enterokinase, or re-cloning using a primer without the FLAG-tag as was done for SEQ ID NO: 47).

### Example 4

### Expression of SUMO-AMP Fusion Proteins

This example describes methods that can be used to generate and express other SUMO-AMP fusion proteins, such as SUMO-defensin (as exemplified by SUMO-HBD-3). One skilled in the art will appreciate that similar methods can be used to produce other SUMO-AMP proteins.

Mouse beta-defensin 3 or human beta-defensin 3 (SEQ ID NO: 9) is N-terminally fused to SUMO protein (SEQ ID NOS: 14 and 15) using the restriction enzymes Nde1 and Xho1, which correspond to the pet28b vector of Novagen. The vector was modified to harbor SUMO as an N-terminal fusion tag and carries a kanamycin resistance cassette. The constructs are transformed into competent BL21 (DElysE) and clones will be checked for positive insertion. The gene will be sequenced and positive clones will be selected for expression. The SUMO-β defensin peptide can be purified and treated with sumoase to release β defensin as described in Example 2. The functionality of the resulting β defensin can be determined as described in Example 3.

While this disclosure has been described with an emphasis upon particular embodiments, it will be obvious to those of ordinary skill in the art that variations of the particular embodiments can be used, and it is intended that the disclosure may be practiced otherwise than as specifically described herein. Features, characteristics, sequences, or examples described in conjunction with a particular aspect, embodiment, or example of the disclosure are to be understood to be applicable to any other aspect, embodiment, or example of the disclosure. Accordingly, this disclosure includes all modifications encompassed within the spirit and scope of the disclosure as defined by the following claims

1. An isolated fusion protein, comprising:
a small ubiquitin related modifier (SUMO) amino acid sequence; and
an antimicrobial peptide (AMP) amino acid sequence.

2. The isolated fusion protein of claim 1, further comprising a purification tag amino acid sequence.

3. The isolated fusion protein of claim 1, wherein the SUMO amino acid sequence is N-terminal to the AMP amino acid sequence.

4. The isolated fusion protein of claim 1, comprising a sequence having at least 70% or at least 95% sequence identity to any of SEQ ID NOS: 22-32, 40, 45, 47, 49, and 51.

5. The isolated fusion protein of claim 1, comprising any of SEQ ID NOS: 22-32, 40, 45, 47, 49, and 51.

6. An isolated nucleic acid molecule encoding the fusion protein of claim 1.

7. A vector comprising the isolated nucleic acid molecule of claim 6.

8. A transgenic cell comprising the vector of claim 7.

9. A method of producing a protein, comprising:
expressing a nucleic acid molecule encoding a fusion protein in a cell, wherein the nucleic acid molecule comprises a nucleic acid molecule encoding small ubiquitin related modifier (SUMO) operably linked to a nucleic acid molecule encoding an antimicrobial peptide (AMP); and
culturing the cell under conditions sufficient for expression of the fusion protein, wherein the presence of SUMO in the fusion protein decreases antimicrobial activity of the AMP of the fusion protein, increases stability of the fusion protein, or combinations thereof.

10. The method of claim 9, further comprising removing the SUMO from the fusion protein, thereby restoring antimicrobial activity to the antimicrobial peptide.

11. The method of claim 9, further comprising isolating the fusion protein.

12. The method of claim 10, wherein the SUMO is removed from the fusion protein in vitro, thereby producing the antimicrobial peptide, and the method comprises incubating the fusion protein in the presence of a protease.

13. The method of claim 10, wherein the SUMO is removed from the fusion protein in vivo, thereby producing the antimicrobial peptide.

14. The method of claim 13, further comprising purifying the antimicrobial peptide.

15. The method of claim 9, wherein the nucleic acid molecule encoding SUMO is operably linked upstream to the nucleic acid molecule encoding the AMP.

16.-18. (canceled)

19. The method of claim 9, wherein the AMP comprises at least one amino acid substitution in a native antimicrobial sequence that increases the antimicrobial activity of the peptide.

20. (canceled)

21. The method of claim 9, wherein the fusion protein further comprises a purification tag.

22. An isolated peptide produced by the method of claim 9.

23. A kit comprising:
the vector of claim 7; and
a protease capable of cleaving the SUMO from the antimicrobial peptide, cells suitable for expression of the vector, or both.

24. A method of treating an infection in a subject, comprising:
administering a therapeutically effective amount of the protein produced by the method of claim 9 to the subject, thereby treating the infection.

25. A kit comprising:
the isolated fusion protein of claim 1; and
a protease capable of cleaving the SUMO from the antimicrobial peptide, cells suitable for expression of the vector, or both.
